(19) United States
(12) Patent Application Publication
(10) Pub. No.: US 2010/0285465 A1
(43) Pub. Date: Nov. 11, 2010

(54) Molecular Typing of Neisseria Strains by Determining the Presence of Genes Involved in Lipooligosaccharide (LOS) Biosynthesis

(75) Inventors: Nathalie Isabelle Devos, Rixensart (BE); Emmanuel Enrico Alexandre Di Paolo, Rixensart (BE); Christiane Feron, Rixensart (BE); Jan Poolman, Rixensart (BE)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: GLAXOSMITHKLINE BIOLOGICALS S.A., Rixensart (BE)

(21) Appl. No.: 12/671,227

(22) PCT Filed: Jul. 31, 2008
(86) PCT No.: PCT/EP2008/060077

§ 371 Date: Jan. 29, 2010

Related U.S. Application Data
(60) Provisional application No. 60/953,551, filed on Aug. 2, 2007.

(51) Int. Cl. C12Q 1/68; C07H 21/04; G01N 33/53

(52) U.S. Cl. 435/6; 536/24.33; 435/7.1

(57) ABSTRACT

The present invention relates to a method of LOS molecular typing of a Neisseria strain by determining the presence of a functional gene and/or gene product of one or more genes involved in the biosynthesis of LOS.

### FIELD OF THE INVENTION

The present invention relates to the field of typing of Neisseria strains. More particularly it relates to a method of classification of Neisseria strains through molecular typing of LOS through the analysis by molecular techniques of genes involved in LOS biosynthesis. This method is useful for the epidemiological classification of circulating Neisseria.

### BACKGROUND OF THE INVENTION

Neisseria meningitidis (meningococcus) is a Gram negative bacterium frequently isolated from the human upper respiratory tract. It is a cause of serious invasive bacterial diseases such as bacteremia and meningitis. The incidence of meningococcal disease shows geographical, seasonal and annual differences (Schwartz, B., Moore, P. S., Broome, C. V.; Clin. Microbiol. Rev. 2 (Supplement), S18-S24, 1989). The bacterium is commonly classified according to the serogroup of its capsular polysaccharide.

Most disease in temperate countries is due to strains of serogroup B and varies in incidence from 1-10/100,000/year total population—sometimes reaching higher values (Kaczmarski, E. B. (1997), Commun. Dis. Rep. Rev. 7: R55-9, 1995; Scholten, R. J. P. M., Bijlmer, H. A., Poolman, J. T. et al. Clin. Infect. Dis. 16: 237-246, 1993; Cruz, C., Pavez, G., Aguilar, E., et al. Epidemiol. Infect. 105: 119-126, 1990).

Epidemics dominated by serogroup A meningococci, mostly in central Africa, sometimes reach incidence levels of up to 1000/100,000/year (Schwartz, B., Moore, P. S., Broome, C. V. Clin. Microbiol. Rev. 2 (Supplement), S18-S24, 1989). Nearly all cases as a whole of meningococcal disease are caused by serogroup A, B, C, W-135 and Y meningococci, and a tetravalent A, C, W-135, Y capsular polysaccharide vaccine is available (Armand, J., Arminjon, F., Mynard, M. C., Lafaix, C., J. Biol. Stand. 10: 335-339, 1982).

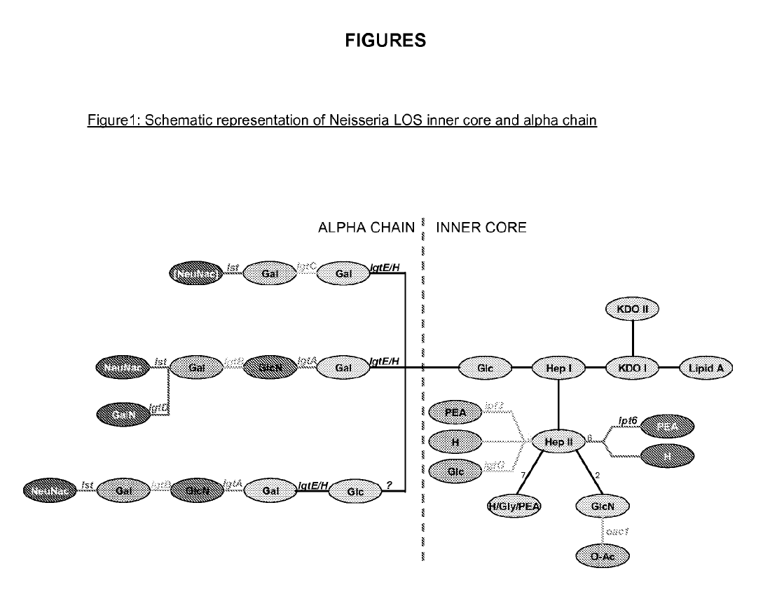

It has been shown that LOS typing is more complex than previously described. In top of the sugar composition of the alpha-chain, “decoration” of heptose II (FIG. 1) seems to have an impact on LOS immunogenicity. PEA (phosphoethanolamine) numbers and positions, presence of Glucose in position 3, presence of Glycine in position 7 and O-acetylation of GlcNac seem to be major determinants of cross-protection.

Analysis of LOS composition by analytical methods is long and tedious. Current methods of LOS typing include immunological or immunotyping. However, this method has its limitations which include: incomplete coverage and difficulties in production and provision of reagents, resulting in an increasing number of isolates not typeable by these means; reliance on expression of the typing target; inconsistent correspondence with the genetic relatedness of isolates, at least partially due to horizontal genetic exchange, combined with the high levels of positive selection experienced by meningococcal surface components; and difficult to apply to non-culture diagnosis and typing (Jolley et al., FEMS Microbiol Review 31:89).

The inventors have developed molecular biology tools applicable rapidly to lots of strain samples, based on the genes involved in LOS biosynthesis, to classify circulating Neisseria strains. This method will be useful for epidemiology studies.

### DESCRIPTION OF THE INVENTION

Reference to “lipooligosaccharide” (or “LOS”) may also be referred to as “lipopolysaccharide” or “LPS”.

The terms “comprising”, “comprise” and “comprises” herein is intended by the inventors to be optionally substitutable with the terms “consisting of”, “consist of”, and “consists of”, respectively, in every instance.

The present inventors have discovered that Neisserial strains can be typed by a method more reliably and easily than current typing methodologies. Current methods rely on immunotyping, which is less reliable method than that of the present inventions. The inventors have shown that using molecular typing techniques, strains can be accurately typed through assessment of the presence and optionally the functionality of the genes involved in the biosynthesis of LOS structures. FIG. 1 below illustrates the genes involved in LOS structure and the position at which they act with in the inner core and the alpha chain of the structure.

The IgtG gene expresses the enzyme adding Glucose in position 3 of Heptose II. This gene is deleted in a number of Neisserial strains, alone or in combination with Ipt6. This gene is phase variable. The Ipt6 gene expresses the enzyme adding PEA in position 6 of Heptose II. The gene is deleted in a number of Neisserial strains (about 50% of a collection of carriage, hyperinvasive and laboratory N.m. strains), alone or in combination with IgtG and is not phase variable.

The genes IgtE (or IgtH), IgtA, IgtC, IgtB, IgtD and Ist are involved in the biosynthesis of the alpha chain.

See FIG. 1 for a schematic representation of the Neisserial LOS inner core and alpha chain.

Accordingly, there is provided a method of LOS molecular typing of a Neisseria strain comprising the steps of a) determining of the presence of a functional gene and/or gene product (+) of at least the Ipt6 and IgtG genes and b) typing the strain as to whether it is either:

| lgtG | lpt6 |
|---|---|
| + | + |
| + | − |
| − | + |
| − | − |

The present invention relates to methods and kits for typing Neisserial strains. These methods can be used in medical and diagnostics, or in research. By “typing” or “differentiation” it is intended the identification of the strain of a bacterium, including identifying that it is distinct from other strains based on the presence and function of specific gene.

By “presence” of a gene it is meant that the whole or part of the specified gene is detectable by methods well known to those skilled in the art. By “functional” it is meant that the gene is capable of transcribing mRNA that when translated results in polypeptide capable of performing the function associated with the gene.

Presence of a functional gene is designated by the symbol “+”, conversely the absence of a gene or the presence of a non-functional gene is designated by the symbol “−”. The presence or absence of functional gene product may also be represented by + or −, respectively.

The Ipt3 gene expresses the enzyme adding PEA in position 3 of Heptose II. The gene is not phase variable and is deleted (partly or completely) in about 14% of a collection of carriage, hyperinvasive and laboratory Neisseria meningitidis strains.

Suitably, one embodiment of the invention provides a method of LOS molecular typing of a Neisseria strain comprising the steps of a) determining of the presence of a functional gene and/or gene product of one at least of the Ipt3, Ipt6 and IgtG genes and b) typing the strain as to whether it is either:

| lpt3 | lgtG | lpt6 |
|---|---|---|
| + | − | − |
| + | + | − |
| + | − | + |
| + | + | + |
| − | − | + |
| − | + | − |
| − | + | + |
| − | − | − |

The enzyme adding PEA in position 7 of Heptose II is unknown. In addition, the enzyme adding Glycine in position 7 of Heptose II is unknown.

The enzyme adding O-Acetyl on GlcNAc was unknown in Neisseria until recently. Recent work by the inventors (see WO2007/144316) showed that the gene involved in LOS acetylation is oac1 (NMB0285 in N. meningitidis B MC58 genome). These results were confirmed by a similar work in Stephen's laboratory (Kalher et al, [2006 J Biol Chem 281(29):19939-48]). oac1 is present in all tested strains and is regulated by phase variation.

Accordingly, in a further embodiment, there is provided a method of LOS molecular typing of a Neisseria strain comprising the steps of a) determining of the presence of a functional gene and/or gene product of at least of the genes Ipt3, Ipt6, IgtG and oac1 and b) typing the strain as to whether it is either:

| lpt3 | lgtG | lpt6 | oac1 |
|---|---|---|---|
| + | − | − | − |
| + | + | − | − |
| + | − | + | − |
| + | + | + | − |
| − | + | − | − |
| − | − | + | − |
| − | + | + | − |
| − | − | − | + |
| − | − | − | − |
| + | − | − | + |
| + | + | − | + |
| + | − | + | + |
| + | + | + | + |
| − | − | + | + |
| − | + | − | + |
| − | + | + | + |

In a further embodiment, there is provided a method of LOS molecular typing of a Neisseria strain comprising the steps of a) determining of the presence of a functional gene and/or gene product of at least of the oac1, Ipt6 and IgtG genes and b) typing the strain as to whether it is either:

| oac1 | lgtG | lpt6 |
|---|---|---|
| + | − | − |
| + | + | − |
| + | − | + |
| + | + | + |
| − | − | + |
| − | + | − |
| − | + | + |
| − | − | − |

Accordingly, in one embodiment of the invention there is provided a method of LOS molecular typing as comprising the following steps:
Performing PCR amplifications from the Neisserial chromosome with probes designed to determine whether one or more of the following functional genes is present in said chromosome: Ipt6 and IgtG; and optionally
Determining whether the IgtG and Ipt6 genes are capable of expressing functional LgtG and Lpt6 proteins, respectively.

Polymerase Chain Reaction (PCR) amplification may be performed from the bacterial chromosome or nucleic acid isolated and or purified from a bacterial culture. Encompassed within the invention but not limited to are PCR reactions carried out on a bacterial cell suspension, crude, isolated or purified chromosomal preparation, using techniques of chromosomal DNA preparation well known to those skilled in the art. The chromosomal preparations encompassed by the present invention may be isolated or substantially purified. By “isolated” or “substantially purified” is intended that the nucleic acid molecules, are substantially or essentially free from components normally found in association with the nucleic acid in its natural state. Such components include other cellular material, culture media from recombinant production, and various chemicals used in chemically synthesizing the nucleic acids.

Methods for designing PCR primers are generally known in the art and are disclosed in Sambrook and Russel, Molecular Cloning: A Laboratory Manual (Cold Harbour Laboratory Press). Known methods of PCR include, but are not limited to, methods using paired primers, nested primers, single specific primers, degenerate primers, gene-specific primers, vector-specific primers, partially mismatched primers, and the like.

With PCR, it is possible to amplify a single copy of a specific target sequence to a level detectable by several different methodologies (e.g., but not limited to, hybridization with a labelled probe; incorporation of biotinylated primers followed by avidin-enzyme conjugate detection; incorporation of 32P-labeled deoxynucleotide triphosphates, such as dCTP or dATP, into the amplified segment, incorporation of a fluorochrome such as etidium bromide, or other commercial compounds). In addition to genomic DNA, any nucleotide sequence can be amplified with the appropriate set of primer molecules. In particular, the amplified segments created by the PCR process itself are, themselves, efficient templates for subsequent PCR amplifications.

Amplification in PCR requires “PCR reagents” or “PCR materials,” which herein are defined as all reagents necessary to carry out amplification except the polymerase, primers, and template. PCR reagents normally include nucleic acid precursors (dCTP, dTTP, etc.), and buffer.

In one embodiment there is provided a method according to the invention wherein the presence of the full length IgtG and Ipt6 genes are assessed. In a further embodiment there is provided a method wherein functionality of the IgtG gene is assessed by the presence or absence of the gene on the Neisserial strain's chromosome. The presence or absence of the gene may be assessed by PCR. Accordingly, there is provided a method wherein the presence or absence of the IgtG gene is determined using any of the following primers: SEQ ID NO 8 or SEQ ID NO 9.

The term “primer” is used herein to mean any single-stranded oligonucleotide sequence capable of being used as a primer in, for example, PCR technology. Thus, a ‘primer’ according to the invention refers to a single-stranded oligonucleotide sequence that is capable of acting as a point of initiation for synthesis of a primer extension product that is complementary to the nucleic acid strand to be copied. The design (length and specific sequence) of the primer will depend on the nature of the DNA and/or RNA targets and on the conditions at which the primer is used (such as temperature and ionic strength).

The primers may consist of the nucleotide sequences shown in SEQ ID NO: 1-40, or may be 10, 15, 20, 25, 30, 35, 40, 45, 50, 75, 100 or more bases which comprise or fall within the sequences of SEQ ID NO: 1-40, provided they are suitable for specifically binding DNA of target loci, under stringent conditions. When needed, slight modifications of the primers or probes in length or in sequence can be carried out to maintain the specificity and sensitivity required under the given circumstances. Probes and/ or primers listed herein may be extended by 1, 2, 3, 4 or 5 nucleotides, for example, in either direction.

As used herein, the term “stringent conditions” means any hybridisation conditions which allow the primers to bind to a specific nucleotide sequence, and not to any other loci on the bacterial chromosome.

In a further embodiment of the invention there is provided a method wherein functionality of the gene Ipt6 is assessed by the presence or absence of the gene. Suitably, there is provided a method wherein the presence or absence of the Ipt6 gene is determined using any of the following primers: SEQ ID NO 4 or SEQ ID NO 5.

Many Neisseria genes are subject to phase variation, which can be defined as the high frequency, reversible on-off switching of gene expression. Phase variation is a feature of genes associated with a variety of meningococcal antigens including LOS.

Several Neisserial LOS biosynthetic genes contain poly(G) tracts of 3 or more bases. These tracts turn the gene off or on. Depending on the on/off status of different LOS biosynthesis genes, different biosynthetic pathways are used to generate different LOS structures.

The genes IgtA, IgtC, IgtD, oac1 and IgtG are subject to phase variation of expression mediated by homopolymeric tracts within their coding regions.

Accordingly, one embodiment of the invention provides a method wherein the functionality of the gene product of the IgtG gene is assessed by sequencing of the gene or part thereof. Preferably, there is provided a method according to the invention wherein the functionality of the gene product of IgtG is assessed by sequencing of the poly-C region. Methods of sequencing are well known to those skilled in the art and include but are not limited to the use of radiolabelled ddNTP terminators or fluorescent labelled terminators with a sequencing reaction.

Suitably, one embodiment of the invention provides a method wherein the functionality of the gene product of IgtG gene is determined using any or both of the following primers: SEQ ID NO:8 or SEQ ID NO:9.

A further embodiment provides a method of LOS molecular typing comprising the following steps:
Performing PCR amplifications from the bacterial chromosome with probes designed to determine whether one or more of the following genes is present in said chromosome: Ipt6, IgtG Ipt3; and optionally
Determining whether the IgtG, Ipt6 and Ipt3 genes are capable of expressing functional LgtG Lpt6 and Lpt3 proteins, respectively.

In embodiment of the invention, there is provided a method as wherein the presence of the full length IgtG, Ipt6 and Ipt3 genes are assessed, preferably a method wherein functionality of the gene Ipt3 is assessed by the presence or absence of the gene, particularly, a method wherein the presence or absence of the Ipt3 gene is determined using any of the following primers: SEQ ID NO 1, SEQ ID NO 2 or SEQ ID NO 3.

In a further embodiment of the invention there is provided a method of LOS molecular typing comprising steps:
Performing PCR amplifications from the bacterial chromosome with probes designed to determine whether one or more of the following genes is present in said chromosome: Ipt6, IgtG, Ipt3 and oac1; and optionally
Determining whether the IgtG, Ipt6, Ipt3 and oac1 genes are capable of expressing functional LgtG, Lpt6, Lpt3 and Oac1 proteins, respectively.

In a further embodiment of the invention, there is provided a method wherein the presence of the full length IgtG, Ipt6, Ipt3 and oac1 genes are assessed.

In a particular embodiment, there is provided a method wherein the presence or absence of the oac1 gene is determined using either or both of the following primers: SEQ ID NO 6 or SEQ ID NO 7 and in a particular embodiment a method of the invention wherein the functionality of the oac1 gene or gene product is assessed by sequencing the oac1 gene or part thereof.

The gene oac1 has 2 phase variable regions, accordingly there is provided a method wherein either or both of the phase variable regions of the oac1 gene are sequenced, particularly a method wherein the poly-G regions of the oac1 gene is sequenced in order to determine the functionality of the oac1 gene. In a specific embodiment, there is provided a method wherein the functionality of the gene product of oac1 gene is determined using any one or more of the following primers: SEQ ID NO:12, SEQ ID NO:13, SEQ ID NO:14, SEQ ID NO:15, SEQ ID NO:16, SEQ ID NO:17 or SEQ ID NO:18.

The presence or absence of a gene or gene loci may be determined by PCR or by other techniques known to the person skilled in the art. Other techniques by which the presence of genes may be assessed include but are not limited to DNA hybridisation, including in situ DNA hybridisation. As used herein, the terms “hybridisation” or “specific hybridisation” means that the primer or probe forms a duplex (double-stranded nucleotide sequence) with part of a target or with the entire region under the experimental conditions used, and that under those conditions the primer or probe does not form a duplex with other regions of the nucleotide sequence present in the sample to be analysed. It should be understood that the primers and probes of the present invention are designed for specific hybridisation to specific genes and may therefore fall entirely within said region or may to a large extent overlap with said region (i.e. form a duplex with nucleotides outside as well as within said region). Accordingly, there is provided a method in which the primers of the inventions may be used during a PCR reaction or DNA hybridisation.

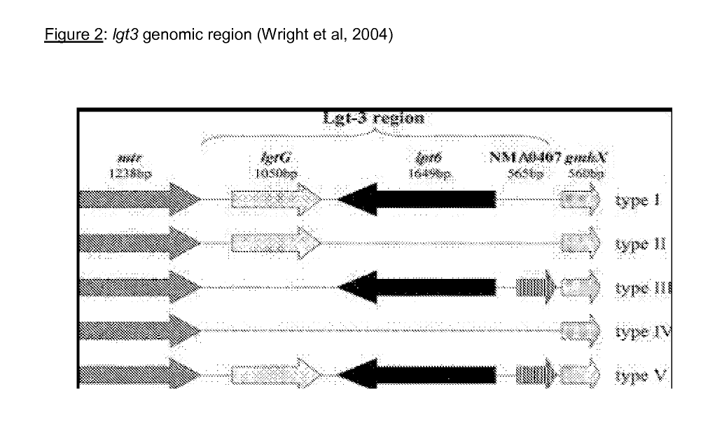

The genes IgtG and Ipt6 are located in the same chromosomal region: Igt3 (FIG. 2). PCR amplification of the Igt3 region could already give information on the presence/absence of Ipt6/IgtG genes. Based on the PCR fragments length obtained, strains have been classified in 5 different groups (type I to V).

Accordingly, in one embodiment of the invention there is provided a method wherein the presence or absence of the IgtG and Ipt6 genes are assessed simultaneously by amplification of the Igt3 chromosomal region. Suitably, there is provided a method wherein a PCR product is generated and the presence or absence of the IgtG and Ipt6 genes are assessed by length of the Igt3 Neisserial chromosomal region. Preferably, there is provided a method wherein the IgtG and Ipt6 genes are assessed using either or both of the following primers: SEQ ID NO 10 or SEQ ID NO11.

In one embodiment of the invention there is provided a method of LOS molecular typing wherein the PCR amplifications are performed using one or more of the following primers: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10 or SEQ ID NO 11, SEQ ID NO:16, SEQ ID NO:17, SEQ ID NO:18, SEQ ID NO:19, SEQ ID NO:20, SEQ ID NO:21 or SEQ ID NO:22

In a further embodiment of the invention there is provided a method of LOS molecular typing wherein the sequencing reactions to determine the functionality of the genes are performed using one or more of the following primers: SEQ ID NO:8, SEQ ID NO:9, SEQ ID NO 12, SEQ ID NO 13, SEQ ID NO 14, SEQ ID NO 15, SEQ ID NO:23, SEQ ID NO:24, SEQ ID NO:25 or SEQ ID NO:26.

In a particular embodiment of the invention, the PCR reactions to detect each of the inner core gene may be performed in a single PCR reaction. Accordingly, one of embodiment of the invention provides a method in which the PCR amplification reactions related to the inner core genes are multiplexed. As used herein, the term “multiplexed” means that a single PCR reaction is performed allowing assessment of the presence or absence of said genes. Primers of the invention are designed so that a single PCR reaction can be performed using specified reaction conditions allowing amplifications of each gene involved in the biosynthesis of the inner core to be amplified without prejudice to the amplification of another gene. Multiplexing allows a faster and easier method of molecular typing of circulating Neisseria strains as described herein.

Accordingly, one embodiment provides a method of LOS typing wherein the multiplex PCR reactions are performed using one or more of the following primers: SEQ ID NO:8, SEQ ID NO: 9, SEQ ID NO:19, SEQ ID NO:20, SEQ ID NO:21, SEQ ID NO:22, SEQ ID NO:23, SEQ ID NO:24, SEQ ID NO:25, SEQ ID NO:26.

Further, there is provided a method wherein the sequencing reactions to determine the functionality of the inner core biosynthetic genes are performed using one or more of the following primers: SEQ ID NO:8, SEQ ID NO:9, SEQ ID NO:23, SEQ ID NO:24, SEQ ID NO:25 or SEQ ID NO:26.

Genes IgtA, IgtB, IgtC, IgtD, IgtE, IgtH and Ist are responsible for the synthesis and thus eventual structure of the α chains. These genes are well known in the art (WO 96/100086, WO 97/47749, Zhu et al., 2006 [Microbiology 152: 123-134]). Genes IgtA, IgtC, and IgtD contain poly(G) tracts. When the number of guanines found in these genes changes during DNA replication, alterations in the coding sequence may occur, making translation of the proteins encoded by these genes susceptible to premature termination. Loss of function of any of these genes effects changes in the structure of LOS.

Accordingly, one embodiment of the invention provides a method in which the LOS is further typed by assessing the outer core structure of the LOS, in particular, a method wherein said method comprises the steps a) determining of the presence of a functional gene and/or gene product of IgtE and b) typing the strain as to whether it is:

lgtE
+
−

In a further embodiment, there is provided a method wherein said method comprises the steps a) determining the presence of a functional gene and/or gene product (+) of IgtH and b) typing the strain as to whether it is:

lgtH
+
−

In a further embodiment, there is provided a method wherein said method comprises the steps a) determining the presence of a functional gene and/or gene product (+) of IgtC and b) typing the strain as to whether it is:

lgtC
+
−

In a further embodiment, there is provided a method wherein said method comprises the steps a) determining the presence of a functional gene and/or gene product (+) of IgtA and b) typing the strain as to whether it is:

lgtA
+
−

In a further embodiment, there is provided a method wherein said method comprises the steps a) determining the presence of a functional gene and/or gene product (+) of IgtB and b) typing the strain as to whether it is:

lgtB
+
−

In a further embodiment, there is provided a method wherein said method comprises the steps a) determining the presence of a functional gene and/or gene product (+) of IgtD and b) typing the strain as to whether it is either:

lgtD
+
−

In a further embodiment, there is provided a method wherein said method comprises the steps a) determining the presence of a functional gene and/or gene product (+) of Ist and b) typing the strain as to whether it is either:

lst
+
−

Suitably, one embodiment of the invention provides a method wherein the presence or absence of any one of the genes that contribute to the structure of the alpha chain is assessed by PCR amplification. A preferred embodiment there is provided a method in which the PCR amplification reactions to determine the absence or presence of genes that encode enzymes that contribute to the alpha chain are multiplexed. Those skilled in the art are aware there are number of methods by which the presence or absence of a gene maybe determined which are also encompassed by the invention. These include but are not limited to hybridisation and in situ hybridisation.

The genes IgtA, IgtC, and IgtD are phase variable. Accordingly there is provided a method of molecular typing wherein the functionality of the gene is determined by sequencing. In a particular embodiment there is provided a method in which the functionality of IgtA, IgtC, and IgtD is determined by sequence of the phase variable regions, in particular the poly(G) regions. Methods of sequencing are well known in the art.

In one embodiment there is provided a method of typing the alpha chain in which the PCR amplification reactions are multiplexed. Specifically, there is provided a method of wherein the multiplex PCR reactions are performed using one or more of the following primers: SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 or SEQ ID NO:40.

Further, there is provided a method of LOS typing wherein the sequencing reactions to determine the functionality of the genes are performed using one or more of the following primers: SEQ ID NO:29, SEQ ID NO:30, SEQ ID NO:37, SEQ ID NO:38, SEQ ID NO:39 or SEQ ID NO:40.

Accordingly, in one embodiment of the invention there is provided a method of LOS molecular typing comprising the steps:
Determining the presence/absence of the IgtG gene or gene product encoded by IgtG;
Determining whether the IgtG gene encodes a functional protein; and
Determining the presence/absence of the Ipt6 gene or gene product encoded by Ipt6.

In a further embodiment, there is provided method as described herein which further comprises the step:
Determining the presence/absence of the Ipt3 gene or gene product encoded by Ipt3.

In a further embodiment there is provided method as described herein which further comprises the steps:
Determining the presence/absence of the oac1 gene or gene encoded by oac1; and
Determining whether the gene oac1 encodes a functional protein.

In a further embodiment there is provided method as described herein further comprising the step:
Determining the presence/absence of the IgtE gene or gene product encoded by IgtE.

In a further embodiment there is provided method as described herein further comprises the step:
Determining the presence/absence of the IgtH gene or gene product encoded by IgtH.

In a further embodiment there is provided method as described herein which further comprises the steps:
Determining the presence/absence of the IgtC gene or gene product encoded by IgtC; and
Determining whether the IgtC gene encodes a functional protein.

In a further embodiment there is provided method as described herein which further comprises the step:
Determining the presence/absence of the IgtB gene or gene product encoded by IgtB.

In a further embodiment there is provided method as described herein which further comprises the steps:
Determining the presence/absence of the IgtA gene or gene product encoded by IgtA; and
Determining whether the IgtA gene encodes a functional protein.

In a further embodiment there is provided method as described herein which further comprises the steps:
Determining the presence/absence of the IgtD gene or gene product encoded by IgtD; and
Determining whether the IgtD gene encodes a functional protein.

In a further embodiment there is provided method as described herein which further comprises the step:
Determining the presence/absence of the Ist gene or gene product encoded by Ist.

Those skilled in the art are aware that there are a variety of methods by which the presence of a functional gene may be assessed. Transcription of a gene results in the production mRNA transcripts. Methods of detecting mRNA are well known to those skilled in the art and are encompassed by the invention. Methods of the invention include but are not limited to hybridization (Northern blot) and Reverse Transcriptase-PCR (RT-PCR). RT-PCR is well known to those skilled in the art.

Accordingly, one embodiment of the invention provides a method of LOS molecular typing comprising the following steps:
Purifying RNA from the Neisserial strain;
Performing RT-PCR amplifications with probes designed to determine whether Ipt6 and IgtG mRNA transcripts are present, and optionally whether Ipt3, oac1, IgtE, IgtD, IgtH, IgtC, IgtB, IgtA or Ist are present;
Producing cDNA from mRNA transcripts; and
Sequencing of cDNA to determine whether a functional IgtG gene is present and optionally one or more of oac1, IgtC, IgtD and IgtA genes.

In a further embodiment of the invention provides a method of LOS molecular typing comprising the following steps:
Purifying RNA from the Neisserial strain;
Performing RT-PCR amplifications with probes designed to determine whether Ipt6, IgtG, IgtE and IgtH mRNA transcripts are present, and optionally whether Ipt3, oac1, IgtD, IgtC, IgtB, IgtA or Ist are present
Producing cDNA from mRNA transcripts; and
Sequencing of cDNA to determine whether a functional IgtG gene is present and optionally one or more of oac1, IgtC, IgtD and IgtA genes.

In further aspect of the invention there is provided a method of LOS molecular typing as claimed in any of the preceding claims comprising the steps:
Producing cell lysate from the strain; and
Detecting the presence of protein encoded by Ipt6 and IgtG; and optionally one or more of Ipt3, oac1, IgtE, IgtH, IgtC, IgtD, IgtB, IgtA and Ist.

In further aspect of the invention there is provided a method of LOS molecular typing as claimed in any of the preceding claims comprising the steps:
Production of cell lysate from the strain; and
Detection of presence of protein encoded by Ipt6, IgtG, IgtE and IgtH; and optionally one or more of Ipt3, oac1, IgtD, IgtC, IgtB, IgtA and Ist.

The present invention additionally provides a kit comprising primers for the molecular typing or diagnosis of Neisseria colonisation carried out according to the methods described herein.

The present invention further provides a kit for the molecular typing or diagnosis of Neisseria colonisation comprising at least primer or set of primers as described herein for the molecular typing or diagnosis of a Neisseria colonisation and/or infection.

In one embodiment of the invention there is provided a method of diagnosis and classification of a Neisseria colonisation and/or infection in a host susceptible to Neisseria colonisation comprising:
Obtaining a biological sample from a host;
   Carrying out the method as described herein, optionally through the use of the kit as described herein.

### Examples

The examples below are carried out using standard techniques, which are well known and routine to those of skill in the art, except where otherwise described in detail. The examples are illustrative, but do not limit the invention.

### Example 1

### Characterization of the LOS of Strains 6275 and C11 by the Ouchterlony Method (Immunotyping), MS/MS Analysis and Molecular Biology Analysis

Summary
Strains 6725 and C11 were immunotyped by immunodiffusion using specific polyclonal antibodies (Ouchterlony method). Their inner core LOS composition was determined by MS/MS analysis. Genes encoding the enzymes involved in LOS inner core decoration were analysed through PCR and sequencing.
Based on MS/MS analysis, strain 6275 possesses two PEA residues on the Hep II. This strain was immunotyped as an L3 strain, though conventional L3 strains have only one PEA (at position 3 on HepII).
Two different compositions of the inner core LOS of strain C11 were observed by MS-MS. One population contains one PEA residue on the Hep II while the second population contains two PEA residues. The strain was immunotyped as an L3 strain with also a very weak reaction with anti-L2 sera.

Introduction

The inner core LOS composition of Neisseria meningitidis LOS appears to be more complex than previously described. Until recently, the inner core LOS was proposed to contain either no PEA or only one PEA residue on the Hep II at position 3 or 6 (or 7). But a new inner core LOS with two PEA residues at position 3 and at position 6 (or 7) was recently described. Because this new LOS structure was described from a strain previously immunotyped as L3, this new LOS structure has been named L3v for L3 variant.

Before the discovery of the L3v structure, epidemiological data based on immunotyping of LOS have shown that around 70% of invasive serogroup B strains were L3 (PEA at position 3) and most of the remaining strains were L2 (PEA at position 6).

Based on bactericidal data obtained with a panel of L3 and L2 strains and sera from animals immunized either with L3 derived blebs or L2 derived blebs we have concluded that only anti-L3 derived sera are able to mediate the complement killing of L3 strains while only anti-L2 sera are able to kill L2 strains. But interestingly bactericidal data obtained on two new L3v strains are not in line with our previous conclusions. Indeed, the complement killing of these two strains is mediated by anti-L2 derived sera but not anti-L3 sera. These two “atypical” strains are the serogroup B strain 6275 and the serogroup C reference strain C11.

In order to understand the divergent results between immunotype and bactericidal results, the inner core composition of strains 6275 and C11 was determined by MS/MS. In addition, immunotyping of these strains was performed using the Ouchterlony method (immunodiffusion using specific polyclonal sera). The presence of functional Ipt3, Ipt6 and IgtG genes were also analysed using molecular biological methods. These genes encode for enzymes responsible for addition on Hep II of a PEA at position 3, a PEA at position 6 and a Glucose at position 3, respectively.

Results

1. Inner Core Composition by MS/MS Analysis

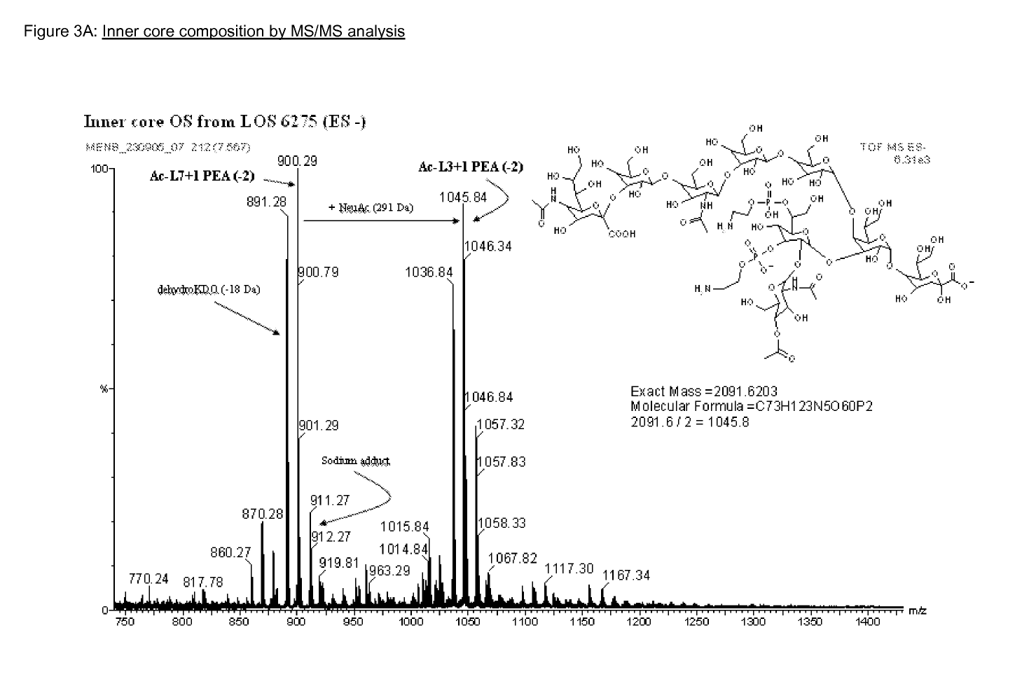

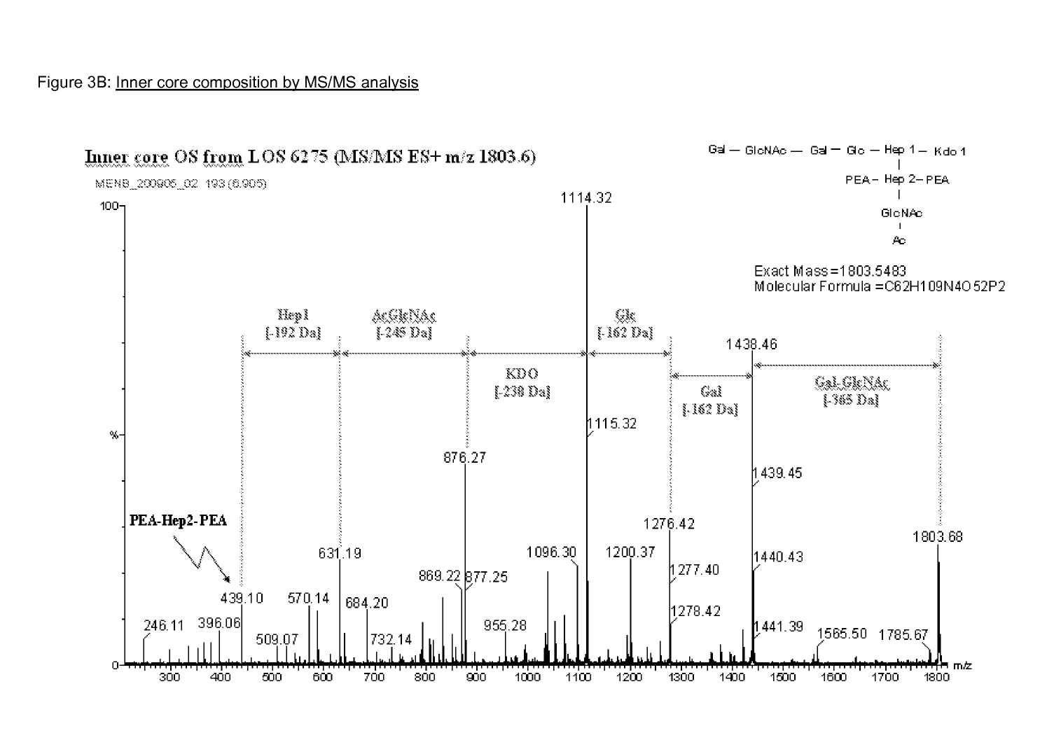

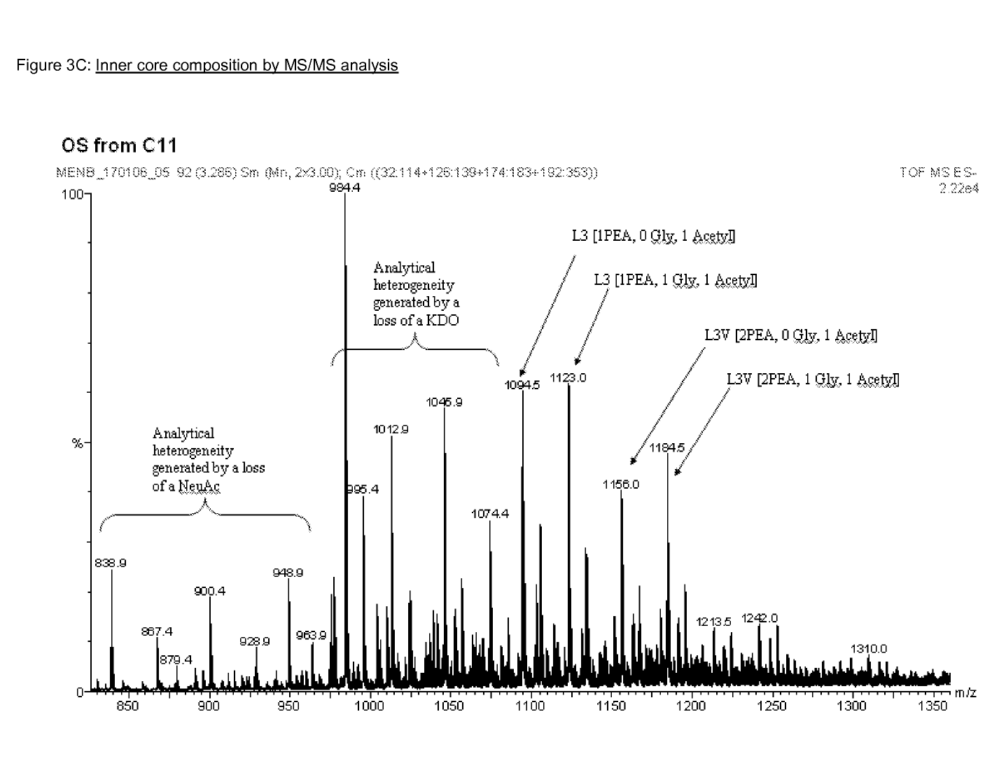

The MS/MS analyses see FIG. 3 show that:
In both strains, the α-chain LOS is the typical LNnT tetrasaccharide described for L2 and L3 strains with a terminal sialic acid group.
The inner core LOS of strain 6275 possesses two PEA residues most probably at positions 3 and 6 but to be confirmed by NMR analysis.
The C11 strain was composed of two different inner cores:
   one population with one PEA (its position on Hep II is not defined, but a weak signal for a glycine on Hep II is observed which excludes a PEA at position 7)
   a second population with two PEA (most probably in position 3 and 6 because a glycine was also detected on this Hep II)
For both strains no glucose is detected on Hep II

2. LOS Immunotyping

The immunotyping was performed using a panel of specific antisera. Only the results obtained with the L3 antiserum and L2 antiserum are described below because the results generated with other antisera (L1, L4, L5 . . . ) were negative for strains 6275 and C11. In these experiments, strains 6275 and C11 currently used at GSK Bio (GSK6275-1&2 and GSK C11) were compared to similar strains conserved in a freezer for more than 20 years at the Amsterdam University (strains Zol 6275 and RIV C11).

The two strains 6275 (Zol 6275 and GSK6275-1&2) have similar behaviour in the immunotyping assay. They react strongly with the anti-L3 serum but not with the anti-L2 serum.

The GSK C11 strain and the RIV C11 strain also display identical results. Both strains are positive with the anti-L3 serum and weakly positive with the anti-L2 serum.

In order to confirm the very weak precipitation obtained with C11 strains and the anti-L2 serum, new immunodiffusion experiments were done in combination with 5 patients' isolates previously typed as L3,2 in 1980. The C11 results show again very weak precipitations with the anti L2 serum while the typing of the 5 disease strains is confirmed.

| Strains | Serogroup | typed 1980 | typed 2006 |
|---|---|---|---|
| 2991 | B | L3,(2) | L3,2 |
| 3072 | W | L3,2 | L3,2 |
| 3146 | C | L3,(2) | L3,(2) |
| 3151 | W | L3,2 | L3,2 |
| 3356 | B | L3,2 | L3,2 |
| GSK C11 | C | | L3,(2) |
| RIV C11 | C | | L3,(2) |

3. Molecular Characterization

The enzyme adding the PEA in position 3 has been identified and is encoded by the gene Ipt3. A PEA in position 3 on Hep II is detected in 70% of hypervirulent N. meningitidis strains and Wright and coworkers detected the Ipt3 gene by PCR in 86% of analyzed strains. This gene is not regulated by phase variation and was found to be partly deleted in various serogroup C and A strains. The addition of a PEA in position 3 has been hypothesized to be in competition with the addition of glucose at the same position. The enzyme involved is encoded by the IgtG gene (see WO04/015099), regulated by phase variation with a polyC tract in the ORF. The hypothesis from the Moxon laboratory is that, if the IgtG gene is present and in frame, a functional enzyme is produced, glucose is added at position 3 and a PEA is not added at that position.

The gene coding for the enzyme adding a PEA in position 6 is Ipt6, located beside the IgtG gene. This gene does not contain regions susceptible to be regulated by phase variation and was detected in 48% of N.m. (Wright et al, 2004). The gene coding for the enzyme adding a Glycine in position 7 on Hep II is not known.

MenB strain 6275 and menC strain C11 were analysed in parallel with corresponding L3 and L2 reference strains. PCR amplification and sequencing experiments were performed:
when a full copy of Ipt3 gene is present (PCR), investigation were pursued on the functionality of the IgtG gene (presence by PCR and sequence of the poly-C stretch in the ORF)
the presence of the Ipt6 gene was assessed by PCR. If a full copy of the gene is present, we will postulate that the strain contains LOS with a PEA in position 6.

### PCR and Sequence Analysis of Ipt3, Ipt6 and IgtG Genes

| | lpt3 | lgtG | | lpt6 |
|---|---|---|---|---|
| | Presence | Presence | Functionality | Presence |
| H44/76 (L3) | + | + | − | − |
| NZ124 (L3) | + | − | − | − |
| B16B6 (L2) | + | + | + | + |
| 760676 (L2) | + | + | + | + |
| 6275 | + | + | +* | + |
| C11 | + | + | +* | + |

Based on those data, menB 6275 and menC C11 strains seem to be related to the L2 immunotype. However, MS/MS analysis shows for both strains two PEA groups and no Glucose. The * indicates that the IgtG gene has 14 rather than 11 (the normal number seen in active genes) consecutive C nucleotides in the phase variable region. Although this means the open reading frame is in frame and thus may produce a functional protein, it is also possible that the addition of an additional Proline residue might disrupt the protein structure and thus its function.

4. Summary

The next table summarises the characterization of different meningococcal strains using different methods

| | | SBA killing | | MS/MS | | Molecular characterization | | |
|---|---|---|---|---|---|---|---|---|
| Strains | Immunotyping | Anti-TrL3 | Anti-TrL2 | PEA | Glc | Ipt3 | Ipt6 | IgtG functional |
| H44/76 | L3 | + | − | 1 | − | + | − | − |
| NZ124 | L3 | + | − | 1 | − | + | − | − |
| 760676 | L2 | − | + | 1 | + | + | + | + |
| B16B6 | L2 | − | + | 1 | + | + | + | + |
| 6275 | L3 | − | + | 2 | − | + | + | +* |
| C11 | L3(2) | − | + | 1&2 | − | + | + | +* |

Discussion

The diversity of the inner core LOS composition is more complex than previously described. Initially, strains without or with one PEA group on Hep II (either on position 3 or 6/7) were depicted but recently strains with two PEA groups were described. Such strains are for example the serogroup B strain 6275 and the serogroup C strain C11.

Surprisingly, immunotyping results and serum bactericidal results obtained with strains 6275 and C11 are not in line. Indeed, these two strains are typed as L3 strains but their killing is mediated by anti-L2 derived blebs sera and not by anti-L3 derived blebs sera (see next example).

A question was raised about the relevance of these strains and whether the presence of two PEA groups could be a laboratory artefact due to successive in-vitro culture passages. We have had the opportunity to compare different seeds of strains 6275 and C11. For each strain a seed currently used by the inventors was compared to an older seed stored for more than 20 years (at the Amsterdam University). For each strain, the two seeds have shown the same behaviour in the Ouchterlony assay suggesting absence of drift at least during these last 20 years. Nevertheless, the inner core LOS composition of the oldest seeds should be determined to confirm the presence of two PEA residues.

According to the literature around 70% of invasive meningococcal strains are L3. The Ouchterlony results obtained with L3v strains suggested that this method does not differentiate between L3 and L3v strains. Therefore, the number of “true L3” strains could be over-estimated. The Ipt3 and Ipt6 genes are responsible for the addition of PEA group at the position 3 and 6 on Hep II respectively. Around 36% of circulating strains contain both genes, 50% possess Ipt3 only and 12% possess Ipt6 only (Wright JC et al., 2004). Therefore potentially 36% of strains could be L3v even if typed as L3. However, recent epidemiological data obtained with a panel of MAbs specific for different inner core LOS structures suggested that less than 2% of strains have two PEA groups on Hep II (Gidney M A J et al., Infect Immun. 2004 72: 559-69). The difference between these two studies (36% versus 2%) could be explained by the higher sensitivity to human complement of strains possessing a PEA group on position 6 (Ram S et al., J Biol Chem. 2003 278:50853-62). Taken together all the data suggest that the majority of invasive strains are “true L3”.

It was suggested that IgtG and Ipt3 compete for the O-3 position of Hep II, with a described bias for the addition of Glc residue over PEA residue (Wright J C et al 2004).

This hypothesis may not be a universal rule as demonstrated by the presence of 2 PEA residues in the inner core LOS of strains 6275 and C11 even in the presence of an functional IgtG gene (unless the in frame IgtG gene with 14 C nucleotides in the phase variable region is not active). In addition to the Moxon laboratory hypothesis another system involved in the regulation/composition of LOS inner-core structure of strain NMB was recently described. This system is the MisR/MisS two component regulatory system (Tzeng Y L et al J Biol Chem. 2004 279:35053-62). In conclusion, the mechanisms involved in the composition of the inner core LOS appear to be multiple and not fully elucidated.

Five strains isolated from patients displayed a surprising LOS immunotype because they show a predominant precipitation with anti-L3 sera but also a weaker precipitation with anti-L2 serum. This is also the case with the strain C11 even if the precipitation with anti-L2 serum is very weak. The MS/MS analysis of the inner core LOS of strain C11 shows also two different inner-core compositions partially in agreement with the co-expression of L3 and L2 LOS. The L3 LOS identified by immunoprecipitation could actually be a L3v LOS (with 2 PEA residues) whilst the L2 LOS should be related to inner core LOS possessing one PEA at position 6 (to be confirmed by NMR analysis) even in absence of detectable Glc which should be present on some LOS molecule due to the detection of an apparently functional IgtG gene in strain C11. Nevertheless, analysis (MS/MS, molecular characterisation and SBA) of the inner core LOS of one or more of those patients isolates should be done to confirm that these L3,2 strains co-express L3v and L2 LOS.

In conclusion, strains possessing two PEA groups on their inner core are immunotyped as L3 strains but this immunotyping is not in agreement with biological reactivity in SBA and analysis of the presence of “functional” IgtG, Ipt3 and Ipt6 genes. Our data indicate that those L3v strains are probably not derived from L3 strains (due to the presence of the Ipt6 gene) and thus should be renamed.

### Example 2

### MS Analysis

This method was used to generate MS data showed in Example 3.

Organic Solvent Pre-Treatment

2-5 g (wet-weight) of cell were transferred in a 15 ml Falcon tube. Cells were washed successively with 2 ml of distilled water, 2 ml of ethanol, 2 ml of acetone and 2 ml of diethyl ether. The bacteria were then thoroughly dried under vacuum.

Phenol/Chloroform/Petroleum Ether Extraction

90 g of solid phenol was dissolved in 11 ml of water. 10 ml of that solution was mixed with 25 ml of chloroform and 40 ml of hexane (or better petroleum ether by 40-60° C.). That mixture must be monophasic and clear. If the mixture was cloudy, it could be made clear by adding solid phenol.

The lyophilised LOS are homogenised in 5 ml of the above extraction mixture with an ultrasonic bath for 5 min. The mixture was then stirred for 1h and centrifuged at 10 000 g for 15 min at 4° C. The supernatant was retrieved in a 15 ml glass tube, and the extraction of the pellet was repeated as described above. The two supernatants were pooled.

The chloroform and the hexane/petroleum ether in the supernatant were removed by evaporation under a flow of nitrogen.

The residual phenol phase was chilled on ice, and the LOS was precipitated by the addition of 6 volumes of cold diethyl ether-acetone (1:5). The precipitate was collected by centrifugation for 15 min at 4° C. The pellet was washed three times with 80% phenol (1 ml) and twice with diethyl ether (1 ml). The pellet was then dried.

LPS Hydrolysis

The dry residue is dissolved in 600 μl of 1% SDS in 100 mM ammonium acetate pH 4.5 by stirring for 15 min. The solution is then heated and stirred for 2 h at 80° C.

OS Purification

The sample must be now processed to remove the detergent. The solution is evaporated to dryness under vacuum.

Removal of the Detergent:

100 μl of water are added +vortex.

1000 μl of cold acidified EtOH (20 ml EtOH+0.5 ml HCl 1N)+vortex

Centrifuge 15 min at 5° C.

Wash three times the pellet with 200 μl of EtOH

Removal of the Lipid A:

Wash again two times the pellet with 250 μl of CHCl3/MeOH (1:1)

The aqueous OS is purified on a SPE carbon cartridge

The OS is then filtered on 0.45μ before the final purification by HPLC SEC (0.05% TFA) and detection at 206 nm. The collected fractions were evaporated to dryness and reconstituted in 10 μl of water/MeOH (1:1, v/v).

Mass Spectrometry

Electrospray mass spectrometry was performed with a QT of II (Micromass, Manchester, UK) equipped with a nanospray ion source. 5 μl of the oligosaccharide solution was placed in a nanospray needle. Samples were run in the negative-ion mode. Mass scale calibration was performed using NaI. Experiments were performed using argon in the collision cell for collisionnal focusing. The integration period was of 2 s. In full scan MS, the cone voltage was of 40 V and the collision energy was of 10 eV.

### Example 3

### Molecular Typing

Molecular Typing was performed on 20 N. meningitidis strains used for in house SBA experiment and our results were correlated with LOS structure defined after Mass Spectrometry analysis.

Strains

MenB strains (H44/76 Norway; S3446; BZ232; M972500687, B16B6 (desensitized), BZ10, 760676 (desensitized), 2986 (desensitized), 6275 and NZ124, 608B and H355), MenA strains (8238, 3125) MenW (3193, S4383 FDA), MenY (S1975 and M010240539), MenC 11 and 19 were obtained from Preclinical Immunology department (C.Tans). All strains were grown on Mueller Hinton, GC or BHI solid medium o/n at 37° C.+5% CO2. Either lysates or genomic DNA purified from cells from ¼ plate resuspended in 30 μl of H2O (QlAamp DNA Mini Kit (QIAGEN CatNo 51304), DNA eluted with 2×200 μl of AE buffer (Qiagen)) were used for direct PCR analysis

PCR Screening

When lysates were used as template DNA, all PCR programs started with a step at 96° C. during 10 minutes.

PCR reactions were done in 50 μl

All primers were used at 10 pm

Ipt3
PCR program: 25 or 30 cycles of (1 min 96° C., 1 min 50° C., 2 or 4 min 72° C.) with a final step of 10 min 72° C.
Primers:

| name | sequence | sens | comment |
|---|---|---|---|
| lpt3-4 | CGGTAAATTTTTATTGTGAAA | sens | −29 to −10 |
| | | | upstream ATG |
| | | | in MC58 |
| lpt3-5 | TGCAACTTTTATTGTGAACT | sens | −29 to −10 |
| | | | upstream ATG |
| | | | in MenAZ2491 |
| | | | and MenC FAM18 |
| lpt3-6 | GGGCATCTTCCCCGACATCAT | reverse | +30 a +54 |
| | | | downstream |
| | | | the STOP |

Expected size: 1578 bp (1270 bp if partial deletion)
Results: all genomic DNAs (except C19 and BZ232) present a band of the correct size (1578 bp)

Ipt6
PCR program: 25 or 30 cycles of (1 min 96° C., 1 min 50° C., 2 or 4 min 72° C.) with a final step of 10 min 72° C.
Primers:

| primers | sequence | sens | comment |
|---|---|---|---|
| lpt6-2 | CAGGTCGCACTACCGCTGGA | sens | +57 to +76 |
| | | | downstream |
| | | | lpt6 ATG |
| lpt6-3 | CTAACGGGCAATTTTCAAAACG | reverse | +1632 to |
| | | | +1653 |
| | | | downstream |
| | | | lpt6 ATG |

Expected size: 1600 bp
Results: all genomic DNAs (except H44/76, NZ124, M972500687, S3446 and MenW S4383) present a band of the correct size.

oac1
PCR program: 25 or 30 cycles of (1 min 96° C., 1 min 50° C., 2 or 4 min 72° C.) with a final step of 10 min 72° C.
Primers:

| primers | sequence | sens | comment |
|---|---|---|---|
| oac1-1: | ATGCAAGCTGTCCGATACAG | sens | on NMB0285 |
| | | | ATG |
| oac1-3: | AGCAGGCGTTCGTGTTTGTG | reverse | −51 to −31 |
| | | | upstream |
| | | | NMB0285 STOP |

Expected size: 1800 bp
Results: all genomic DNAs present a band of the correct size.

IgtG
PCR program: 25 or 30 cycles of (1 min 96° C., 1 min 50° C., 30 sec or 2 min 72° C.) with a final step of 10 min 72° C.
Primers:

| name | sequence | sens | comment |
|---|---|---|---|
| lgtGc | gattatggcgcattccca | sens | +408 to +425 |
| | | | downstream lgtG |
| | | | ATG |
| lgtGd | ggtcgatgggaacaggaa | reverse | +581 to +598 |
| | | | downstream lgtG |
| | | | ATG |

Expected size: 200 bp
Results: all genomic DNAs (except NZ124, S3446 and MenA3125) present a band of the correct size.

Lgt3 Genomic Region
PCR program: 30 cycles of (1 min 96° C., 1 min 60° C., 5 min 72° C.) with a final step of 10 min 72° C.
Primers:

| name | sequence | sense | comment |
|---|---|---|---|
| EDPlpt3-1 | aacatcgccgcacaggtattgag | sense | see Zhu |
| | | | et al. |
| | | | (2002) |
| EDPlpt3-2 | acgccgtcgcggtcgagaat | reverse | see Zhu |
| | | | et al. |
| | | | (2002) |

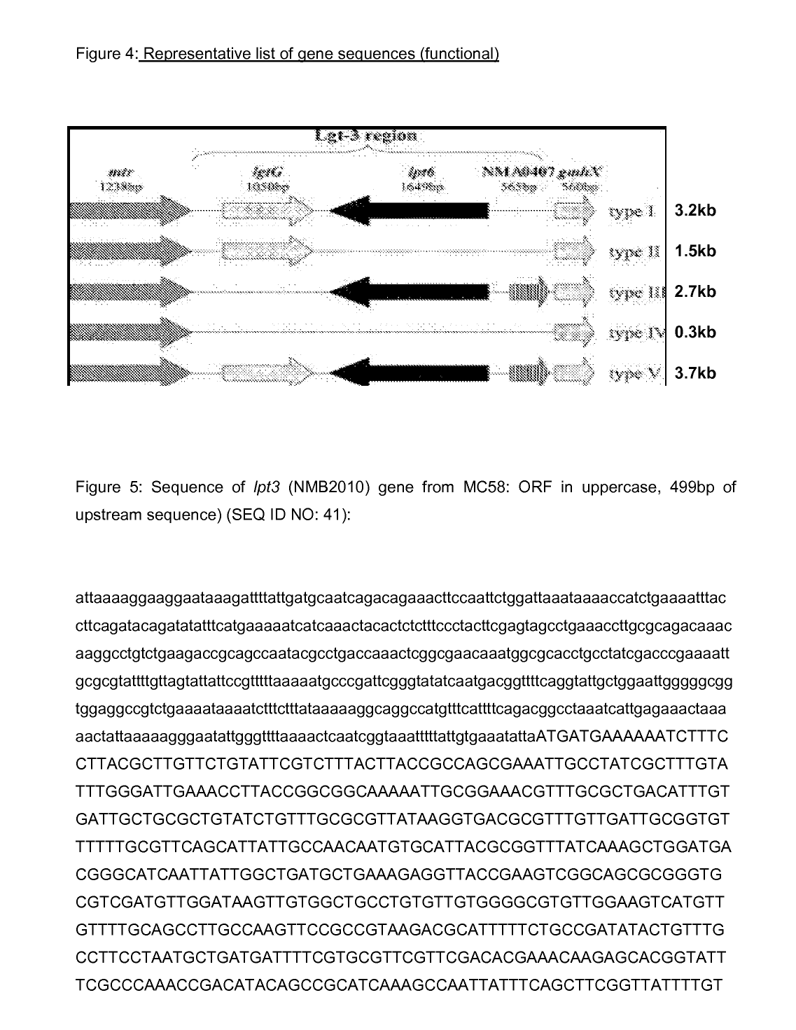

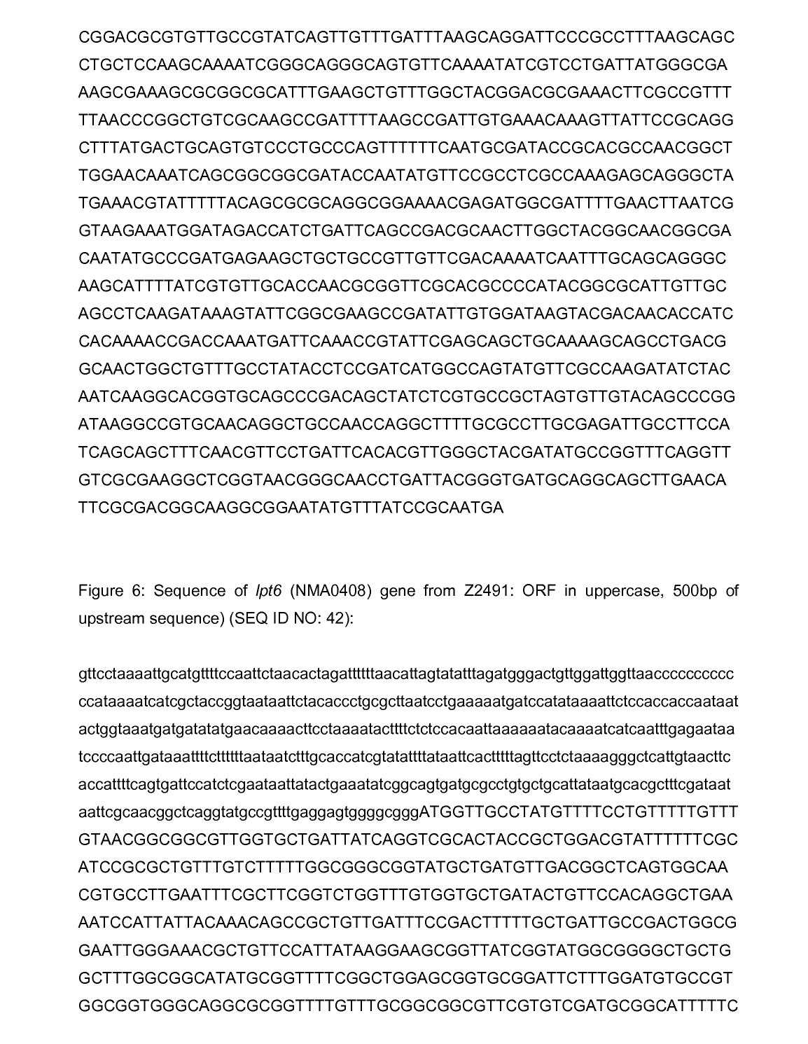

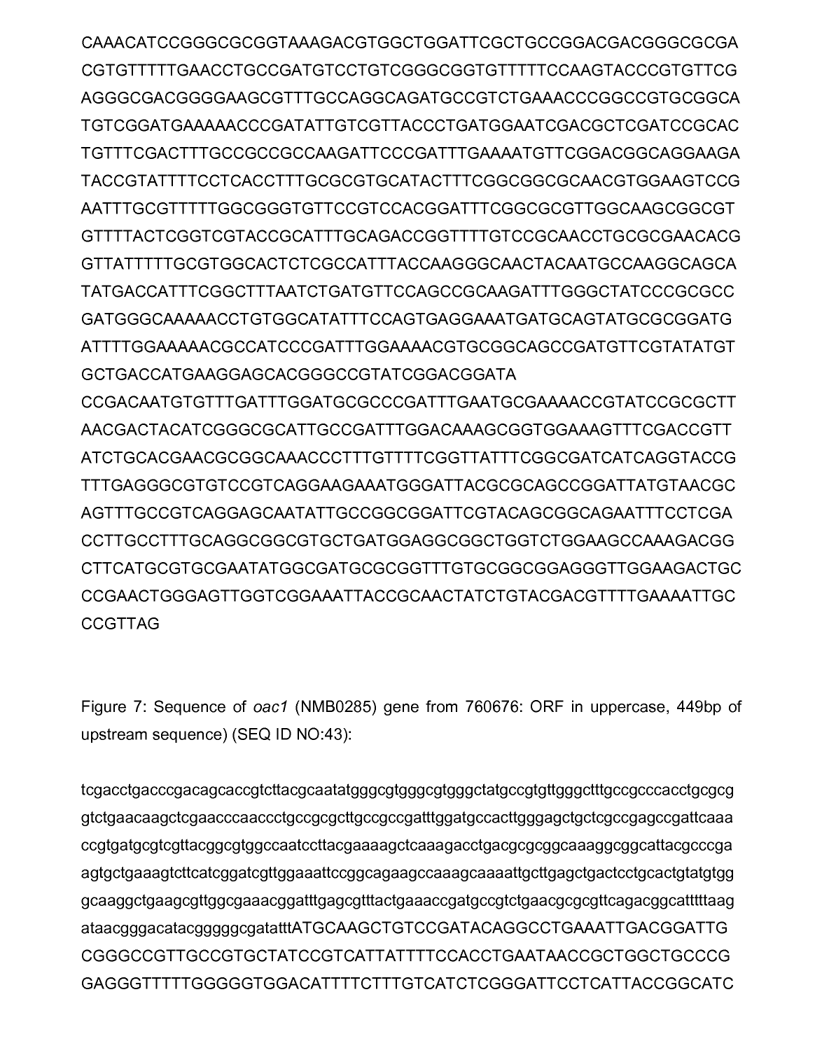

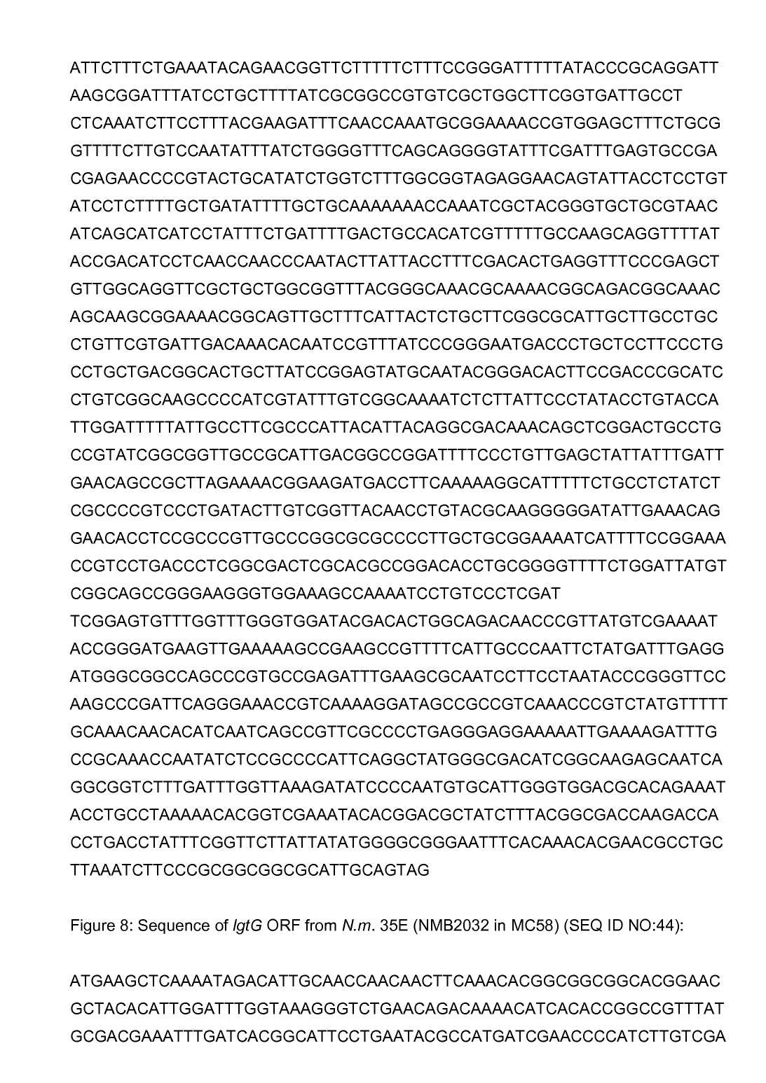

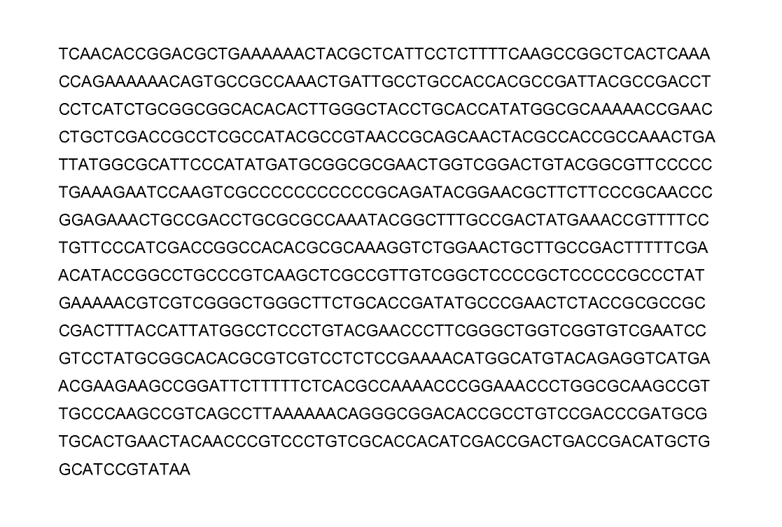

Expected size: size differs according to presence/absence of IgtG, Ipt6 and NMA407 genes (see FIG. 4)
Results: all genomic DNAs present a band ranging from 0.3 to 3.7 kb (see Figure).

DNA Sequencing

IgtG
DNA template: IgtG or Igt3 PCR fragments purified with the “High Pure PCR product purification” Kit (Roche)
Sequencing program: 25 cycles of (30 sec 96° C., 15 sec 50° C., 2 min 30 sec 60° C.)
Primers:

| | | | |
|---|---|---|---|
| name | sequence | sens | comment |

| | | | |
|---|---|---|---|
| lgtGc | gattatggcgcattccca | sens | +408 to +425 |
| | | | downstream lgtG |
| | | | ATG |
| lgtGd | ggtcgatgggaacaggaa | reverse | +581 to +598 |
| | | | downstream lgtG |
| | | | ATG |

Results: gene IN phase for 760676 (11C), 2986(11C), B16B6 (11C), BZ232(11C), 6275(14C) and Cl 1(14C). Out of phase (9,10, 12, 13 or 15C) for all others

oac1 (NMB2085)
oac1 is regulated by at least two distinct phase variable regions (IN phase: 4G and 5G) starting at base 354 and 1136, respectively (NMB0285 in N.menB MC58 genome).
DNA template: oac1 PCR fragment purified with the “High Pure PCR product purification” Kit (Roche)
Sequencing program: 25 cycles of (30 sec 96° C., 15 sec 50° C., 2 min 30 sec 60° C.)
Primers:

| | | | |
|---|---|---|---|
| Primers | Sequence | Sens | Comment |

| | | | |
|---|---|---|---|
| oac1-2 | GAAATTGACGGATTGCGGGCC | sens | +25 to +45 downstream NMB0285 - first polyG |
| oac1-seq20 | TCAGTGTCGAAAGGTAATAAG | reverse | +593 to +613 donwtream NMB0285 - first polyG |
| EDPoac3 | CCTGTTCGTGATTGACAAACAC | sens | +741 to +762 downstream NMB0285 ATG - second polyG |
| oac1-5 | CACTCCGAATCGAGGGACAGG | reverse | +1299 to +1319 downstream NMB0285 - second polyG |

Results: gene IN phase (5G) for all strains except H44/76 and M972500687 (6G)

Selective PCR on oac1 Phase Variable Region (nucl. 1136-1140)
PCR amplification was performed using primers EDPoac15, EDPoac16 and oac1-4 (see table hereafter). The forward primers (EDPoac15, EDPoac16) contain the phase variation region with respectively 5 and 6 G. PCR amplification program differs in the annealing temperature to allow amplification (700 bp) if perfect match only.
Primers:

| Primers | Sequence | Sens | Comment |
|---|---|---|---|
| EDPoac15 | CCTGTACGC | sens | Hybridizes on the IN |
| | AAGGGGGA | | phase of the second |
| | | | polyG |
| EDPoac16 | CTGTACGCA | sens | Hybridizes on the OUT |
| | GGGGGGA | | OF phase of the second |
| | | | polyG |
| oac1-4 | AATTCCCGCCC | reverse | +1796 to +1817 |
| | CATATAATAAG | | downstream |
| | | | NMB0285 ATG |

PCR program: 30 cycles of (1 min 96° C., 1 min 65.5° C. (5G) or 64° C. (6G), 90 sec 72° C.) with a final step of 10 min at 72° C.

Under the conditions performed above and using alternative conditions and primers (e.g. different length, sequence complementary to the poly G region in the middle of primer) selective PCR on both the first and second poly G phase variable regions of the oac1 gene was unable to differentiate between the phase variants. Sequencing of the phase variable regions, however, was successfully able to differentiate between those strains that were IN or OUT of phase and this strategy was relied on for the analysis below.

Data Summary and Correlation with Mass Spectrometry Data

The table 2 shows the presence and functionality of Ipt3, Ipt6, IgtG and oac1 genes in several N. meningitidis strains. Results obtained by MS analysis are in grey. Roman numbers in brackets are the Igt3 region group as defined by Wright et al, 2004.

TABLE 2
Data summary and correlation with Mass Spectrometry data

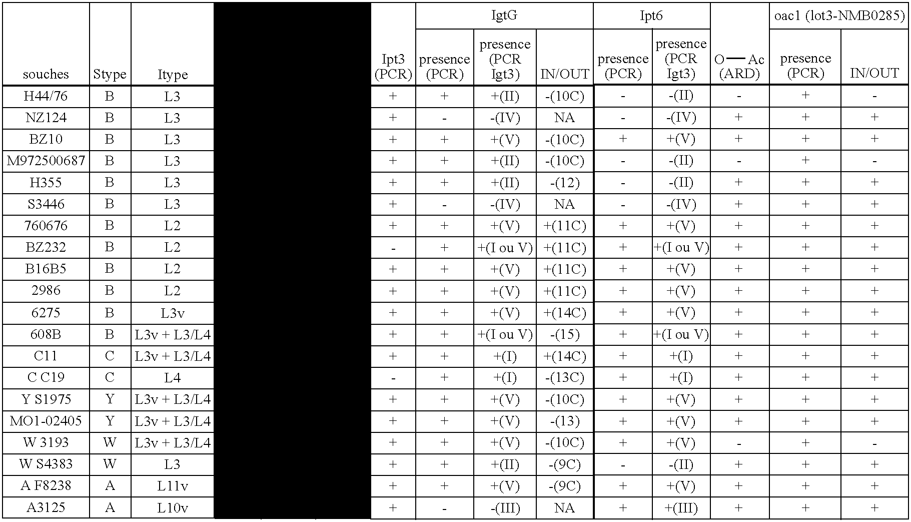

Validation of the Method

95% (19 out of 20 strains) correlation between analytical data and the molecular typing concerning PEA position and number.

100% (20 out of 20 strains) correlation between analytical data and the molecular typing concerning the LOS O-acetylation.

90% (18 out of 20 strains) correlation between analytical data and the molecular typing concerning glucose presence/absence. In the majority of the strains adding a glucose in position 3 of Heptose II, the C-stretch in the IgtG gene is 11. With 14C, the gene is in phase, but the enzyme possess one additional proline which may be deleterious for the enzyme activity, explaining why in 2 strains analysed, MS and Molecular Typing are not aligned. The correlation between both techniques could rise to 100% if the hypothesis of the inactivation of IgtG with the additional proline is confirmed.

### Example 4

### PCR Multiplexing for LOS Inner Core Typing

The aim of the PCR multiplexing is to decrease the workload of the PCR LOS typing approach.
PCR reactions were done in 50 μl
All primers were used at 10 pm

Template DNA

Either lysates or genomic DNA purified from cells from ¼ plate resuspended in 30 μl of H₂O (QIAamp DNA Mini Kit (QIAGEN CatNo 51304), DNA eluted with 2×200 μl of AE buffer (Qiagen)) were used for direct PCR analysis

Primers and PCR Amplification
Primers were designed to amplify fragments of 200-400 bp. Fragments corresponding to the different genes to be analysed (i.e IgtG, Ipt3, Ipt6 and oac1) must be different in size.
PCR amplification of genes with phase variable regions (i.e. IgtG and oac1) encompasses those regions that have to be sequenced.
Two set of primers have to be designed for oac1 as two phase variable regions have to be analysed.

| | | | | | Expected |
|---|---|---|---|---|---|
| Gene | Primers | Sequence | Sens | Comment | size |
| lgtG | lgtGc | GATTATGGCGCATTCCCA | Sens | +408 to +425 downstream lgtG ATG | 190 bp |
| | lgtGd | GGTCGATGGGAACAGGAA | Reverse | +581 to +598 downstream lgtG ATG | |
| lpt6 | EDPlp6-1s | TGCCAAGGCAGCATATGAC | Sens | +954 to +972 downstream lpt6 ATG | 238 bp |
| | EDPlpt6-2as | ACACATTGTCGGTATCCGTC | Reverse | +1173 to +1192 downstream lpt6 ATG | |
| lpt3 | EDPlpt3-17s | GGCGAAGCCGATATTGTG | Sens | +1153 to 1170 downstream lpt3 ATG | 302 bp |
| | EDPlpt3-18as | CGTGTGAATCAGGAACGTTG | Reverse | +1436 to +1455 downstream lpt3 ATG | |
| oac1 | EDPoac32s | CTTCGGTGATTGCCTCTCA | Sens | +257 to +275 downstream oac1 ATG | 370 bp |
| polyG1 | EDPoac33as | ACAGCTCGGGAAACCTCA | Reverse | +610 to +627 downstream oac1 ATG | |
| oac1 | EDPoac37s | ATTACAGGCGACAAACAGCTC | Sens | +945 to +965 downstream oac1 ATG | 421 bp |
| polyG2 | EDPoac38as | TTCGACATAACGGGTTGTCTG | Reverse | +1346 to +1366 downstream oac1 ATG | |

Qiagen Multiplex PCR kit was used to perform PCR reaction
PCR program: 15 min at 95° C., 30 cycles of (30 sec 94° C., 90 sec 50° C., 40 sec 72° C.) with a final step of 10 min 72° C.

DNA Sequencing
DNA template: PCR multiplexing fragments purified with the “High Pure PCR Product Purification” kit (Roche)
Sequencing program: 25 cycles of (30 sec 96° C., 15 sec 50° C., 2 min 30 sec 60° C.)
Primers:

IgtG

| name | sequence | sens | comment |
|---|---|---|---|
| lgtGc | gattatggcgcattccca | sens | +408 to +425 |
| | | | downstream lgtG |
| | | | ATG |
| lgtGd | ggtcgatgggaacaggaa | reverse | +581 to +598 |
| | | | downstream lgtG |
| | | | ATG |

oac1

| Primers | Sequence | Sens | Comment |
|---|---|---|---|
| EDPoac32s | CTTCGGTGA | Sens | +257 to +275 |
| | TTGCCTCTCA | | downstream oac1 ATG |
| EDPoac33as | ACAGCTCGG | Reverse | +610 to +627 |
| | GAAACCTCA | | downstream oac1 ATG |
| EDPoac37s | ATTACAGGCG | Sens | +945 to +965 |
| | ACAAACAGCTC | | downstream oac1 ATG |
| EDPoac38as | TTCGACATAAC | Reverse | +1346 to +1366 |
| | GGGTTGTCTG | | downstream oac1 ATG |

### Example 5

### PCR Multiplexing for Alpha Chain Typing

Strains

MenB strains (H44/76 Norway; S3446; BZ232; B16B6 (desensitized), BZ10, 760676 (desensitized), 2986 (desensitized), 6275, NZ124, 2991, 3356, DE10302_{—}05, DE10672_{—}06 and H355), MenA strains (F8238, 3125 and 3048) MenW (3151, 3193, S4383), MenY S1975, MenC C11, 126E, DE9842 and C19 were obtained from our Preclinical Immunology department. All strains were grown on Mueller Hinton, GC or BHI solid medium o/n at 37° C.+5% CO2.

Template DNA

Either lysates or genomic DNA purified from cells from ¼ plate resuspended in 30 μl of H2O (QIAamp DNA Mini Kit (QIAGEN CatNo 51304), DNA eluted with 2×200 μl of AE buffer (Qiagen)) were used for direct PCR analysis

Primers and PCR Amplification
The aim of the PCR multiplexing is to decrease the workload of the PCR LOS typing approach.
PCR reactions were done in 50 μl
All primers were used at 10 pm
Primers were designed to amplify fragments of 200-400 bp. Fragments corresponding to the different genes to be analysed (i.e IgtA, IgtB, IgtC, IgtE, IgtH and Ist) must be different in size.
PCR amplification of genes with phase variable regions (i.e. IgtA and IgtC) encompasses those regions that have to be sequenced.
Qiagen Multiplex PCR kit was used to perform PCR reaction.
PCR program: 15 min at 95° C., 30 cycles of (30 sec 94° C., 90 sec 50° C., 40 sec 72° C.) with a final step of 10 min 72° C.

The table below shows the primer sets used to amplify fragments of each of the genes IgtB, IgtA, IgtH, IgtE, Ist and IgtC. There are 2 primer sets for IgtC: EDPIgtC1s/EDPIgtC2as; and EDPIgtC5s/EDPIgtC8as. Under the conditions by which the PCR reactions were performed the primer set EDPIgtC5s/EDPIgtC8as provided the optimal amplification of a defined IgtC gene fragment.

| | | | | | Expected |
|---|---|---|---|---|---|
| | Primers | Sequence | | Comment | size |
| lgtB | EDPlgtB1s | CTGCATTATGCCAAGTTTCAC | sens | +625 to +645 downstream lgtB | 152 bp |
| | | | | ATG - based on Zhu et al. (2002) | |
| | EDPlgtB2as | TTCCCTGCTGATTTTGGTC | reverse | +759 to +777 downstream lgtB ATG | |
| lgtA | EDPlgtA1s | ACGGTACGCTTGCCATTG | sens | +179 to +196 downstream lgtA ATG | 223 bp |
| | EDPlgtA2as | GCTGCGGTCTTTTTCCATC | reverse | +384 to +402 downstream lgtA ATG | |
| lgtH | EDPlgtH1s | CGTTTTGCCGTCTGAACG | sens | +513 to +530 downstream lgtH ATG | 267 bp |
| | EDPlgtH2as | GAGGTGTTTTTCCAATTTTTTC | reverse | +759 to +780 downstream lgtH ATG | |
| lgtE | EDPlgtE1s | TTGCGAAAGTTATTGTCAGACC | sens | +374 to +395 downstream lgtE ATG | 360 bp |
| | EDPlgtE2as | AAGTCAAACATCACCTTCAACG | reverse | +713 to +734 downstream lgtE ATG | |
| lst | EDPlst7s | AAGCGGATTTATTTGGCAAG | sens | +408 to +425 downstream lst ATG | 407 bp |
| | EDPlst8as | AAATCTTTTGCCGCCTTTTC | reverse | +796 to +815 downstream lst ATG | |
| lgtC | EDPlgtC1s | CCGATACGGAAATCAGGTTC | sens | +80 to +99 doqnstream lgtC ATG | 497 bp |
| | EDPlgtC2as | CAAAATGTCCTGATCCTGATATTG | reverse | +554 to +577 downstream lgtC ATG | |
| lgtC | EDPlgtC5s | ATACGGAAATCAGGTTCCACG | sens | +83 to +103 downstream lgtC ATG | 486 bp |
| | EDPlgtC8as | CCTGATCCTGATATTGCATCAC | reverse | +548 to +569 downstream lgtC ATG | |

DNA Sequencing
DNA template: PCR multiplexing fragments purified with the “High Pure PCR Product Purification” kit (Roche)
   Sequencing program: 25 cycles of (30 sec 96° C., 15 sec 50° C., 2 min 30 sec 60° C.)
   Primers:

IgtA

| Primers | Sequence | Sens | Comment |
|---|---|---|---|
| EDPlgtA1s | ACGGTACGCT | Sens | +179 to +196 |
| | TGCCATTG | | downstream lgtA ATG |
| EDPlgtA2as | GCTGCGGTC | Reverse | +384 to +402 |
| | TTTTTCCATC | | downstream lgtA ATG |

IgtC

| Primers | Sequence | Sens | Comment |
|---|---|---|---|
| EDPlgtC5s | ATACGGAAATC | sens | +83 to +103 |
| | AGGTTCCACG | | downstream lgtC ATG |
| EDPlgtC8as | CCTGATCCTGA | reverse | +548 to +569 |
| | TATTGCATCAC | | downstream lgtC ATG |

LOS Analysis by Tricine Gel Electrophoresis and Silver Staining

1 to 5 μl of cell lysate was analyzed by electrophoresis on 16% Tricine gel. After silver nitrate staining, the LOS band size was compared to a control L8 and L3/L7 LOS together with the Novex Sharp Pre-stained Protein Molecular weight marker (Invitrogen).

Data Summary and Correlation with Mass Spectrometry Data

The table 3 shows the presence and functionality of IgtA, IgtB, IgtC, IgtE, IgtH and Ist genes in 25 N. meningitidis strains determined by molecular typing and the LOS size estimation observed by electrophoresis and silver staining. Results obtained by MS analysis are in grey.

TABLE 3
data summary and correlation with MS data

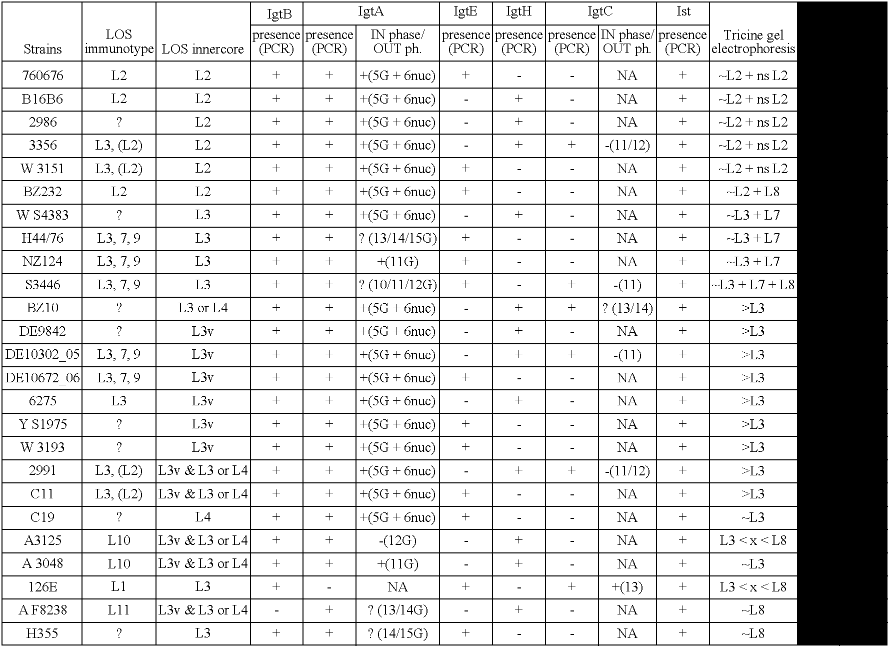

*from Wakarchuk et al, 1998

Conclusion:

Sialylation: Ist gene is reported to be present in all Neisseria meningitidis strains and this is confirmed by our analysis but expression of this gene does not seem to be regulated by phase variation. However, in strains whose LOS displays the correct acceptor site for sialic acid, variable level of sialylation is observed, both by electrophoresis and MS analysis. Thus, analysis of Ist gene by molecular typing will not inform on the LOS sialylation level but could serve as positive control in the multiplexing analysis.

Our multiplex PCR analysis also confirm that each Neisseria meningitidis genome contains IgtE or IgtH gene and that these genes are mutually exclusive.

LOS Tricine gel analysis is in accordance with MS analysis except for 3 strains:

S3436 and BZ232 LOS was determined by MS as mainly L3 type while the main form observed by Silver staining is L8 although L3 and L7 LOS are also observed for S3436 and L3 for BZ232. In strain S3436, the sequence of the phase variable G stretch of IgtA gene could not be determined for sure due to either technical problem or a mixed population of cells. At the opposite, in strain BZ232, the G number in the G stretch keeps the ORF frame and the expressed LOS should indeed be mainly L3 type.

A3048 LOS alpha chain was determined by MS to contain only 2 Hexoses while silver staining analysis reveals a LOS band with a size equivalent to L3 LOS and thus containing 3 Hexoses in the alpha chain. In this strain, the molecular typing analysis is in accordance with the size observed by electrophoresis as the IgtA and IgtB genes are present and functional.

In 20/25 strains (80%), a structure of the alpha chain could be proposed after molecular typing analysis. In the 5 remaining strains, the high number of G (up to 15) in the phase variation stretch impairs the determination of the frame without ambiguity.

Among the 20 strains for which a structure could be proposed, 19 (95%) were in line with the data of MS and/or Tricine gel analysis: all of them but 1 possess functional IgtA and IgtB gene in frame and display a L3/L7-like alpha chain.

Strain 126E genome does not contain IgtA gene but a functional IgtC gene and thus possess the predicted L1 alpha chain composed of 2 Hexoses.

Strain A3125 genome contain IgtB and IgtA genes but the IgtA gene is out of phase. The predicted structure is a L8-like alpha chain with 1 Hexose. However, both MS and Silver staining analysis suggest an alpha chain composed of 2 Hexoses.

To summarize, in our panel of 25 strains, the alpha chain structure is predicted correctly in 76%, unpredictable (L3/L7 or L8) in 20% and incorrectly predicted in 4%.

TABLE 4
Primers used during LOS typing.

| SEQ ID NO | Sequence | Name |
|---|---|---|
| 1 | CGGTAAATTTTTATTGTGAAA | lpt3-4 |
| 2 | TGCAACTTTTATTGTGAACT | lpt3-5 |
| 3 | GGGCATCTTCCCCGACATCAT | lpt3-6 |
| 4 | CAGGTCGCACTACCGCTGGA | lpt6-2 |
| 5 | CTAACGGGCAATTTTCAAAACG | lpt6-3 |
| 6 | ATGCAAGCTGTCCGATACAG | oac1-1 |
| 7 | AGCAGGCGTTCGTGTTTGTG | oac1-3 |
| 8 | GATTATGGCGCATTCCCA | lgtGc |
| 9 | GGTCGATGGGAACAGGAA | lgtGd |
| 10 | AACATCGCCGCACAGGTATTGAG | EDPlpt3-1 |
| 11 | ACGCCGTCGCGGTCGAGAAT | EDPlpt3-2 |
| 12 | GAAATTGACGGATTGCGGGCC | oac1-2 |
| 13 | TCAGTGTCGAAAGGTAATAAG | oac1-seq20 |
| 14 | CCTGTTCGTGATTGACAAACAC | EDPoac3 |
| 15 | CACTCCGAATCGAGGGACAGG | oac1-5 |
| 16 | CCTGTACGCAAGGGGGA | EDPoac15 |
| 17 | CTGTACGCAAGGGGGGA | EDPoac16 |
| 18 | AATTCCCGCCCCATATAATAAG | oac1-4 |
| 19 | TGCCAAGGCAGCATATGAC | EDPlpt6-1s |
| 20 | ACACATTGTCGGTATCCGTC | EDPlpt6-2as |
| 21 | GGCGAAGCCGATATTGTG | EDPlpt3-17s |
| 22 | CGTGTGAATCAGGAACGTTG | EDPlpt3-18as |
| 23 | CTTCGGTGATTGCCTCTCA | EDPoac32s |
| 24 | ACAGCTCGGGAAACCTCA | EDPoac33as |
| 25 | ATTACAGGCGACAAACAGCTC | EDPoac37s |
| 26 | TTCGACATAACGGGTTGTCTG | EDPoac38as |
| 27 | CTGCATTATGCCAAGTTTCAC | EDPlgtB1s |
| 28 | TTCCCTGCTGATTTTGGTC | EDPlgtB2as |
| 29 | ACGGTACGCTTGCCATTG | EDPlgtA1s |
| 30 | GCTGCGGTCTTTTTCCATC | EDPlgtA2as |
| 31 | CGTTTTGCCGTCTGAACG | EDPlgtH1s |
| 32 | GAGGTGTTTTTCCAATTTTTTC | EDPlgtH2as |
| 33 | TTGCGAAAGTTATTGTCAGACC | EDPlgtE1s |
| 34 | AAGTCAAACATCACCTTCAACG | EDPlgtE2as |
| 35 | AAGCGGATTTATTTGGCAAG | EDPlst7s |
| 36 | AAATCTTTTGCCGCCTTTTC | EDPlst8as |
| 37 | CCGATACGGAAATCAGGTTC | EDPlgtC1s |
| 38 | CAAAATGTCCTGATCCTGATATTG | EDPlgtC2as |
| 39 | ATACGGAAATCAGGTTCCACG | EDPlgtC5s |
| 40 | CCTGATCCTGATATTGCATCAC | EDPlgtC8as |

### Example 6

### Classification

Inner Core “Decorations”:

Based on the typing as shown in the Examples above strains maybe typed using the classification method as shown in Table 4A, wherein the inner core of strains are typed by the Ipt3, IgtG, Ipt6 and oac1 genes/gene products.

TABLE 4A

| | Ipt3 | IgtG | Ipt6 | oac1 | strains |
|---|---|---|---|---|---|
| 1.1 | + | − | − | − | H44/76, M685 |
| 1.2 | + | − | − | + | NZ124, S3446, S4383, H355 |
| 2.1 | +/− | + | + | − | |
| 2.2 | +/− | + | + | + | 760676, 2986, B16B6, BZ232 |
| 3.1 | + | − | + | − | W3193 |
| 3.2 | + | − | + | + | 608B, BZ10, 6275, C11, S1975, |
| | | | | | MO1-0240539, F8238, 3125 |
| 4.1 | − | − | + | − | |
| 4.2 | − | − | + | + | C19 |
| 5.1 | +/− | + | − | − | |
| 5.2 | +/− | + | − | + | |
| 6.1 | − | − | − | − | |
| 6.2 | − | − | − | + | |

When the gene responsible for adding the glycine at position 7 on Hep II of the inner core is identified, the strain maybe be typed according to the classification in Table 4B

TABLE 4B

| | Ipt3 | IgtG | Ipt6 | oac1 | glyt | strains |
|---|---|---|---|---|---|---|
| 1.1.1 | + | − | − | − | + | H44/76, M685 |
| 1.1.2 | + | − | − | − | − | |
| 1.2.1 | + | − | − | + | + | NZ124, S3446, S4383, H355 |
| 1.2.2 | + | − | − | + | − | |
| 2.1.1 | +/− | + | + | − | + | |
| 2.1.2 | +/− | + | + | − | − | |
| 2.2.1 | +/− | + | + | + | + | 760676, 2986, B16B6, BZ232 |
| 2.2.2 | +/− | + | + | + | − | |
| 3.1.1 | + | − | + | − | + | W3193 |
| 3.1.2 | + | − | + | − | − | |
| 3.2.1 | + | − | + | + | + | 608B, BZ10, 6275, C11, |
| | | | | | | S1975, MO1-0240539, |
| | | | | | | F8238, 3125 |
| 3.2.2 | + | − | + | + | − | |
| 4.1.1 | − | − | + | − | + | |
| 4.1.2 | − | − | + | − | − | |
| 4.2.1 | − | − | + | + | + | C19 |
| 4.2.2 | − | − | + | + | − | |
| 5.1.1 | +/− | + | − | − | + | |
| 5.1.2 | +/− | + | − | − | − | |
| 5.2.1 | +/− | + | − | + | + | |
| 5.2.2 | +/− | + | − | + | − | |
| 6.1.1 | − | − | − | − | − | |
| 6.1.2 | − | − | − | − | + | |
| 6.2.1 | − | − | − | + | − | |
| 6.2.2 | − | − | − | + | + | |

When the gene that is responsible for the addition of the PEA at position 7 on the HepII of the inner core is identified, this may be used to further type this structure, the classification in Table 4C may be used.

TABLE 4C

| | Ipt3 | IgtG | Ipt6 | oac1 | Ipt7 | strains |
|---|---|---|---|---|---|---|
| 1.1.1 | + | − | − | − | − | H44/76, M685 |
| 1.1.2 | + | − | − | − | + | |
| 1.2.1 | + | − | − | + | − | NZ124, S3446, S4383, H355 |
| 1.2.2 | + | − | − | + | + | |
| 2.1.1 | +/− | + | + | − | − | |
| 2.1.2 | +/− | + | + | − | + | |
| 2.2.1 | +/− | + | + | + | − | 760676, 2986, B16B6, BZ232 |
| 2.2.2 | +/− | + | + | + | + | |
| 3.1.1 | + | − | + | − | − | W3193 |
| 3.1.2 | + | − | + | − | + | |
| 3.2.1 | + | − | + | + | − | 608B, BZ10, 6275, C11, |
| | | | | | | S1975, MO1-0240539, |
| | | | | | | F8238, 3125 |
| 3.2.2 | + | − | + | + | + | |
| 4.1.1 | − | − | + | − | − | |
| 4.1.2 | − | − | + | − | + | |
| 4.2.1 | − | − | + | + | − | C19 |
| 4.2.2 | − | − | + | + | + | |
| 5.1.1 | +/− | + | − | − | − | |
| 5.1.2 | +/− | + | − | − | + | |
| 5.2.1 | +/− | + | − | + | − | |
| 5.2.2 | +/− | + | − | + | + | |
| 6.1.1 | − | − | − | − | + | |
| 6.1.2 | − | − | − | − | − | |
| 6.2.1 | − | − | − | + | + | |
| 6.2.2 | − | − | − | + | − | |

When both the genes responsible for the glycine and PEA at position 7 (Ipt7) are identified, this may be used to type the Neisserial strains further and the classification in Table 4D may be used.

TABLE 4D

| | Ipt3 | IgtG | Ipt6 | oac1 | glyt | Ipt7 | strains |
|---|---|---|---|---|---|---|---|
| 1.1.1 | + | − | − | − | + | − | H44/76, M685 |
| 1.1.2 | + | − | − | − | − | + | |
| 1.1.3 | + | − | − | − | − | − | |
| 1.2.1 | + | − | − | + | + | − | NZ124, S3446, |
| | | | | | | | S4383, H355 |
| 1.2.2 | + | − | − | + | − | + | |
| 1.2.3 | + | − | − | + | − | − | |
| 2.1.1 | +/− | + | + | − | + | − | |
| 2.1.2 | +/− | + | + | − | − | + | |
| 2.1.3 | +/− | + | + | − | − | − | |
| 2.2.1 | +/− | + | + | + | + | − | 760676, 2986, |
| | | | | | | | B16B6, BZ232 |
| 2.2.2 | +/− | + | + | + | − | + | |
| 2.2.3 | +/− | + | + | + | − | − | |
| 3.1.1 | + | − | + | − | + | − | W3193 |
| 3.1.2 | + | − | + | − | − | + | |
| 3.1.3 | + | − | + | − | − | − | |
| 3.2.1 | + | − | + | + | + | − | 608B, BZ10, 6275, |
| | | | | | | | C11, S1975, |
| | | | | | | | MO1-0240539, |
| | | | | | | | F8238, 3125 |
| 3.2.2 | + | − | + | + | − | + | |
| 3.2.3 | + | − | + | + | − | − | |
| 4.1.1 | − | − | + | − | + | − | |
| 4.1.2 | − | − | + | − | − | + | |
| 4.1.3 | − | − | + | − | − | − | |
| 4.2.1 | − | − | + | + | + | − | C19 |
| 4.2.2 | − | − | + | + | − | + | |
| 4.2.3 | − | − | + | + | − | − | |
| 5.1.1 | +/− | + | − | − | + | − | |
| 5.1.2 | +/− | + | − | − | − | + | |
| 5.1.3 | +/− | + | − | − | − | − | |
| 5.2.1 | +/− | + | − | + | + | − | |
| 5.2.2 | +/− | + | − | + | − | + | |
| 5.2.3 | +/− | + | − | + | − | − | |
| 6.1.1 | − | − | − | − | − | + | |
| 6.1.2 | − | − | − | − | + | − | |
| 6.1.3 | − | − | − | − | − | − | |
| 6.2.1 | − | − | − | + | − | + | |
| 6.2.2 | − | − | − | + | + | − | |
| 6.2.3 | − | − | − | + | − | − | |

Alpha Chain Typing

This inner core typing could be completed with the genes involved in the biosyntesis of the alpha chain (IgtA, IgtB, IgtE, IgtH, IgtC, IgtD, Ist) and could constitute a separate classification.

TABLE 5

| | Ist | IgtA | IgtB | IgtC | IgtE/IgtH |
|---|---|---|---|---|---|
| α | − | +/− | +/− | +/− | − |
| β | − | − | +/− | − | + |
| χ | + | − | +/− | − | + |
| δ | − | − | +/− | + | + |
| ε | + | − | +/− | + | + |
| φ | +/− | + | − | + | + |
| γ | − | + | + | + | + |
| η | + | + | + | + | + |
| ι | +/− | + | − | − | + |
| Φ | − | + | + | − | + |
| κ | + | + | + | − | + |

Following this new typing:
H44/76 will be classified B: 1.1.1: κ
NZ124 will be classified B: 1.2.1: κ
6275 will be classified B: 3.2.1: κ

1-66. (canceled)

67. A method of lipooligosaccharide (LOS) molecular typing of a Neisseria strain comprising the steps of
a) determining the presence of a functional gene (+) and/or gene product (+) of at least the Ipt6 and IgtG genes and
b) typing the strain as to whether it is either:

| lgtG | lpt6 |
|---|---|
| + | + |
| + | − |
| − | + |
| − | − |

68. The method of claim 67 wherein
step a) further comprises determining the presence of a functional gene (+) and/or gene product (+) of Ipt3; and
step b) further comprises typing the strain as to whether it is either:

| lpt3 | lgtG | lpt6 |
|---|---|---|
| + | − | − |
| + | + | − |
| + | − | + |
| + | + | + |
| − | − | + |
| − | + | − |
| − | + | + |
| − | − | − |

69. The method of claim 67 wherein
step a) further comprises determining the presence of a functional gene (+) and/or gene product (+) of Ipt3 and oac1; and
step b) further comprises typing the strain as to whether it is either:

| lpt3 | lgtG | lpt6 | oac1 |
|---|---|---|---|
| + | − | − | − |
| + | + | − | − |
| + | − | + | − |
| + | + | + | − |
| − | + | − | − |
| − | − | + | − |
| − | + | + | − |
| − | − | − | − |
| − | − | − | + |
| + | − | − | + |
| + | + | − | + |
| + | − | + | + |
| + | + | + | + |
| − | − | + | + |
| − | + | − | + |
| − | + | + | + |

70. The method of claim 67 wherein
step a) further comprises determining the presence of a functional gene (+) and/or gene product (+) of oac1; and
step b) further comprises typing the strain as to whether it is either:

| oac1 | lgtG | lpt6 |
|---|---|---|
| + | − | − |
| + | + | − |
| + | − | + |
| + | + | + |
| − | − | + |
| − | + | − |
| − | + | + |
| − | − | − |

71. A method of lipooligosaccharide (LOS) molecular typing of a Neisseria strain comprising the steps of
a) performing PCR amplifications from the Neisserial strain's chromosome with probes designed to determine whether the following genes are present in said chromosome: Ipt6 and IgtG;
b) determining the presence of a functional gene (+) and/or gene product (+) of at least the Ipt6 and IgtG genes, and
c) typing the strain as to whether it is either:

| lgtG | lpt6 |
|---|---|
| + | + |
| + | − |
| − | + |
| − | − |

72. The method of claim 67 wherein in step a) the presence of the full length IgtG and Ipt6 genes in the Neisserial strain's chromosome is assessed.

73. The method of claim 67 wherein in step a) the presence of the Ipt6 gene in the Neisserial strain's chromosome is determined by PCR.

74. The method of claim 68 wherein step a) comprises performing PCR amplifications from the Neisserial strain's chromosome with probes designed to determine whether the Ipt3 gene is present in said chromosome.

75. The method of claim 68 wherein the presence of the full length IgtG, Ipt6 and Ipt3 genes in the Neisserial strain's chromosome is assessed

76. The method of claim 69 wherein step a) comprises performing PCR amplifications from the Neisserial strain's chromosome with probes designed to determine whether one or more of all of the following genes are present in said chromosome: Ipt6, IgtG, Ipt3 and oac1.

77. The method of claim 70 wherein step a) comprises performing PCR amplifications from the Neisserial strain's chromosome with probes designed to determine whether the oac1 gene is present in said chromosome.

78. The method of claim 69 wherein in step a) the presence of the full length IgtG, Ipt6, Ipt3 and oac1 genes is assessed.

79. The method of claim 68 wherein the presence of the IgtG and Igt6 genes are assessed simultaneously by amplification of the Igt3 region of the Neisserial chromosome.

80. The method of claim 78 wherein a PCR product is generated and the presence of the IgtG and Igt6 genes are assessed by length of the Igt3 Neisserial chromosome region.

81. The method of claim 67 in which the LOS is further typed by assessing the outer core structure of the LOS.

82. The method of claim 79 wherein
step a) further comprises determining the presence of a functional gene and/or gene product (+) of IgtH; and
step b) further comprises typing the strain as to whether it is either:
lgtH
+
−

83. The method of LOS molecular typing of claim 67 wherein step a) comprises producing cell lysate from the Neisserial strain and detecting the presence of protein encoded by Ipt6 and IgtG.

84. A kit comprising primers for the molecular typing or diagnosis of Neisseria colonisation carried out according to the methods according to claim 67.

85. A method of diagnosis and classification of a Neisseria colonization in a host susceptible to Neisseria colonization comprising obtaining a biological sample from a host and carrying out the method as claimed in claim 67.


SEQUENCE LISTING (44 sequences)

SEQ ID NO: 1 - DNA, 21 bp, Artificial Sequence
  lpt3-4 primer
cggtaaatttttattgtgaaa

SEQ ID NO: 2 - DNA, 20 bp, Artificial Sequence
  lpt3-5 primer
tgcaacttttattgtgaact

SEQ ID NO: 3 - DNA, 21 bp, Artificial Sequence
  lpt3-6 primer
gggcatcttccccgacatcat

SEQ ID NO: 4 - DNA, 20 bp, Artificial Sequence
  lpt6-2 primer
caggtcgcactaccgctgga

SEQ ID NO: 5 - DNA, 22 bp, Artificial Sequence
  lpt6-3 primer
ctaacgggcaattttcaaaacg

SEQ ID NO: 6 - DNA, 20 bp, Artificial Sequence
  oac1-1 primer
atgcaagctgtccgatacag

SEQ ID NO: 7 - DNA, 20 bp, Artificial Sequence
  oac1-3 primer
agcaggcgttcgtgtttgtg

SEQ ID NO: 8 - DNA, 18 bp, Artificial Sequence
  lgtGc primer
gattatggcgcattccca

SEQ ID NO: 9 - DNA, 18 bp, Artificial Sequence
  lgtGd primer
ggtcgatgggaacaggaa

SEQ ID NO: 10 - DNA, 23 bp, Artificial Sequence
  EDPlpt3-1 primer
aacatcgccgcacaggtattgag

SEQ ID NO: 11 - DNA, 20 bp, Artificial Sequence
  EDPlpt3-2 primer
acgccgtcgcggtcgagaat

SEQ ID NO: 12 - DNA, 21 bp, Artificial Sequence
  oac1-2 primer
gaaattgacggattgcgggcc

SEQ ID NO: 13 - DNA, 21 bp, Artificial Sequence
  oac1-seq20 primer
tcagtgtcgaaaggtaataag

SEQ ID NO: 14 - DNA, 22 bp, Artificial Sequence
  EDPoac3 primer
cctgttcgtgattgacaaacac

SEQ ID NO: 15 - DNA, 21 bp, Artificial Sequence
  oac1-5 primer
cactccgaatcgagggacagg

SEQ ID NO: 16 - DNA, 17 bp, Artificial Sequence
  EDPoac15 primer
cctgtacgcaaggggga

SEQ ID NO: 17 - DNA, 17 bp, Artificial Sequence
  EDPoac16 primer
ctgtacgcaagggggga

SEQ ID NO: 18 - DNA, 22 bp, Artificial Sequence
  oac1-4 primer
aattcccgccccatataataag

SEQ ID NO: 19 - DNA, 19 bp, Artificial Sequence
  EDPlpt6-1s primer
tgccaaggcagcatatgac

SEQ ID NO: 20 - DNA, 20 bp, Artificial Sequence
  EDPlpt6-2as primer
acacattgtcggtatccgtc

SEQ ID NO: 21 - DNA, 18 bp, Artificial Sequence
  EDPlpt3-17s primer
ggcgaagccgatattgtg

SEQ ID NO: 22 - DNA, 20 bp, Artificial Sequence
  EDPlpt3-18as primer
cgtgtgaatcaggaacgttg

SEQ ID NO: 23 - DNA, 19 bp, Artificial Sequence
  EDPoac32s primer
cttcggtgattgcctctca

SEQ ID NO: 24 - DNA, 18 bp, Artificial Sequence
  EDPoac33as primer
acagctcgggaaacctca

SEQ ID NO: 25 - DNA, 21 bp, Artificial Sequence
  EDPoac37s primer
attacaggcgacaaacagctc

SEQ ID NO: 26 - DNA, 21 bp, Artificial Sequence
  EDPoac38as primer
ttcgacataacgggttgtctg

SEQ ID NO: 27 - DNA, 21 bp, Artificial Sequence
  EDPlgtB1s primer
ctgcattatgccaagtttcac

SEQ ID NO: 28 - DNA, 19 bp, Artificial Sequence
  EDPlgtB2as primer
ttccctgctgattttggtc

SEQ ID NO: 29 - DNA, 18 bp, Artificial Sequence
  EDPlgtA1s primer
acggtacgcttgccattg

SEQ ID NO: 30 - DNA, 19 bp, Artificial Sequence
  EDPlgtA2as primer
gctgcggtctttttccatc

SEQ ID NO: 31 - DNA, 18 bp, Artificial Sequence
  EDPlgtH1s primer
cgttttgccgtctgaacg

SEQ ID NO: 32 - DNA, 22 bp, Artificial Sequence
  EDPlgtH2as primer
gaggtgtttttccaattttttc

SEQ ID NO: 33 - DNA, 22 bp, Artificial Sequence
  EDPlgtE1s primer
ttgcgaaagttattgtcagacc

SEQ ID NO: 34 - DNA, 22 bp, Artificial Sequence
  EDPlgtE2as primer
aagtcaaacatcaccttcaacg

SEQ ID NO: 35 - DNA, 20 bp, Artificial Sequence
  EDPlst7s primer
aagcggatttatttggcaag

SEQ ID NO: 36 - DNA, 20 bp, Artificial Sequence
  EDPlst8as primer
aaatcttttgccgccttttc

SEQ ID NO: 37 - DNA, 20 bp, Artificial Sequence
  EDPlgtC1s primer
ccgatacggaaatcaggttc

SEQ ID NO: 38 - DNA, 24 bp, Artificial Sequence
  EDPlgtC2as primer
caaaatgtcctgatcctgatattg

SEQ ID NO: 39 - DNA, 21 bp, Artificial Sequence
  EDPlgtC5s primer
atacggaaatcaggttccacg

SEQ ID NO: 40 - DNA, 22 bp, Artificial Sequence
  EDPlgtC8as primer
cctgatcctgatattgcatcac

SEQ ID NO: 41 - DNA, 2077 bp, N. meningitidis
attaaaaggaaggaataaagattttattgatgcaatcagacagaaacttccaattctggattaaataaaaccatctgaaaatttaccttcagatacagatatatttcatgaaaaatcatcaaactacactctctttccctacttcgagtagcctgaaaccttgcgcagacaaacaaggcctgtctgaagaccgcagccaatacgcctgaccaaactcggcgaacaaatggcgcacctgcctatcgacccgaaaattgcgcgtattttgttagtattattccgtttttaaaaatgcccgattcgggtatatcaatgacggttttcaggtattgctggaattgggggcggtggaggccgtctgaaaataaaatctttctttataaaaaggcaggccatgtttcattttcagacggcctaaatcattgagaaactaaaaactattaaaaagggaatattgggttttaaaactcaatcggtaaatttttattgtgaaatattaatgatgaaaaaatctttccttacgcttgttctgtattcgtctttacttaccgccagcgaaattgcctatcgctttgtatttgggattgaaaccttaccggcggcaaaaattgcggaaacgtttgcgctgacatttgtgattgctgcgctgtatctgtttgcgcgttataaggtgacgcgtttgttgattgcggtgttttttgcgttcagcattattgccaacaatgtgcattacgcggtttatcaaagctggatgacgggcatcaattattggctgatgctgaaagaggttaccgaagtcggcagcgcgggtgcgtcgatgttggataagttgtggctgcctgtgttgtggggcgtgttggaagtcatgttgttttgcagccttgccaagttccgccgtaagacgcatttttctgccgatatactgtttgccttcctaatgctgatgattttcgtgcgttcgttcgacacgaaacaagagcacggtatttcgcccaaaccgacatacagccgcatcaaagccaattatttcagcttcggttattttgtcggacgcgtgttgccgtatcagttgtttgatttaagcaggattcccgcctttaagcagcctgctccaagcaaaatcgggcagggcagtgttcaaaatatcgtcctgattatgggcgaaagcgaaagcgcggcgcatttgaagctgtttggctacggacgcgaaacttcgccgtttttaacccggctgtcgcaagccgattttaagccgattgtgaaacaaagttattccgcaggctttatgactgcagtgtccctgcccagttttttcaatgcgataccgcacgccaacggcttggaacaaatcagcggcggcgataccaatatgttccgcctcgccaaagagcagggctatgaaacgtatttttacagcgcgcaggcggaaaacgagatggcgattttgaacttaatcggtaagaaatggatagaccatctgattcagccgacgcaacttggctacggcaacggcgacaatatgcccgatgagaagctgctgccgttgttcgacaaaatcaatttgcagcagggcaagcattttatcgtgttgcaccaacgcggttcgcacgccccatacggcgcattgttgcagcctcaagataaagtattcggcgaagccgatattgtggataagtacgacaacaccatccacaaaaccgaccaaatgattcaaaccgtattcgagcagctgcaaaagcagcctgacggcaactggctgtttgcctatacctccgatcatggccagtatgttcgccaagatatctacaatcaaggcacggtgcagcccgacagctatctcgtgccgctagtgttgtacagcccggataaggccgtgcaacaggctgccaaccaggcttttgcgccttgcgagattgccttccatcagcagctttcaacgttcctgattcacacgttgggctacgatatgccggtttcaggttgtcgcgaaggctcggtaacgggcaacctgattacgggtgatgcaggcagcttgaacattcgcgacggcaaggcggaatatgtttatccgcaatga

SEQ ID NO: 42 - DNA, 2153 bp, N. meningitidis
gttcctaaaattgcatgttttccaattctaacactagattttttaacattagtatatttagatgggactgttggattggttaaccccccccccccataaaatcatcgctaccggtaataattctacaccctgcgcttaatcctgaaaaatgatccatataaaattctccaccaccaataatactggtaaatgatgatatatgaacaaaacttcctaaaatacttttctctccacaattaaaaaatacaaaatcatcaatttgagaataatccccaattgataaattttcttttttaataatctttgcaccatcgtatattttataattcactttttagttcctctaaaagggctcattgtaacttcaccattttcagtgattccatctcgaataattatactgaaatatcggcagtgatgcgcctgtgctgcattataatgcacgctttcgataataattcgcaacggctcaggtatgccgttttgaggagtggggcgggatggttgcctatgttttcctgtttttgtttgtaacggcggcgttggtgctgattatcaggtcgcactaccgctggacgtattttttcgcatccgcgctgtttgtctttttggcgggcggtatgctgatgttgacggctcagtggcaacgtgccttgaatttcgcttcggtctggtttgtggtgctgatactgttccacaggctgaaaatccattattacaaacagccgctgttgatttccgactttttgctgattgccgactggcggaattgggaaacgctgttccattataaggaagcggttatcggtatggcggggctgctggctttggcggcatatgcggttttcggctggagcggtgcggattctttggatgtgccgtggcggtgggcaggcgcggttttgtttgcggcggcgttcgtgtcgatgcggcatttttccaaacatccgggcgcggtaaagacgtggctggattcgctgccggacgacgggcgcgacgtgtttttgaacctgccgatgtcctgtcgggcggtgtttttccaagtacccgtgttcgagggcgacggggaagcgtttgccaggcagatgccgtctgaaacccggccgtgcggcatgtcggatgaaaaacccgatattgtcgttaccctgatggaatcgacgctcgatccgcactgtttcgactttgccgccgccaagattcccgatttgaaaatgttcggacggcaggaagataccgtattttcctcacctttgcgcgtgcatactttcggcggcgcaacgtggaagtccgaatttgcgtttttggcgggtgttccgtccacggatttcggcgcgttggcaagcggcgtgttttactcggtcgtaccgcatttgcagaccggttttgtccgcaacctgcgcgaacacggttatttttgcgtggcactctcgccatttaccaagggcaactacaatgccaaggcagcatatgaccatttcggctttaatctgatgttccagccgcaagatttgggctatcccgcgccgatgggcaaaaacctgtggcatatttccagtgaggaaatgatgcagtatgcgcggatgattttggaaaaacgccatcccgatttggaaaacgtgcggcagccgatgttcgtatatgtgctgaccatgaaggagcacgggccgtatcggacggataccgacaatgtgtttgatttggatgcgcccgatttgaatgcgaaaaccgtatccgcgcttaacgactacatcgggcgcattgccgatttggacaaagcggtggaaagtttcgaccgttatctgcacgaacgcggcaaaccctttgttttcggttatttcggcgatcatcaggtaccgtttgagggcgtgtccgtcaggaagaaatgggattacgcgcagccggattatgtaacgcagtttgccgtcaggagcaatattgccggcggattcgtacagcggcagaatttcctcgaccttgcctttgcaggcggcgtgctgatggaggcggctggtctggaagccaaagacggcttcatgcgtgcgaatatggcgatgcgcggtttgtgcggcggagggttggaagactgcccgaactgggagttggtcggaaattaccgcaactatctgtacgacgttttgaaaattgcccgttag

SEQ ID NO: 43 - DNA, 2318 bp, N. meningitidis
tcgacctgacccgacagcaccgtcttacgcaatatgggcgtgggcgtgggctatgccgtgttgggctttgccgcccacctgcgcggtctgaacaagctcgaacccaaccctgccgcgcttgccgccgatttggatgccacttgggagctgctcgccgagccgattcaaaccgtgatgcgtcgttacggcgtggccaatccttacgaaaagctcaaagacctgacgcgcggcaaaggcggcattacgcccgaagtgctgaaagtcttcatcggatcgttggaaattccggcagaagccaaagcaaaattgcttgagctgactcctgcactgtatgtgggcaaggctgaagcgttggcgaaacggatttgagcgtttactgaaaccgatgccgtctgaacgcgcgttcagacggcatttttaagataacgggacatacgggggcgatatttatgcaagctgtccgatacaggcctgaaattgacggattgcgggccgttgccgtgctatccgtcattattttccacctgaataaccgctggctgcccggagggtttttgggggtggacattttctttgtcatctcgggattcctcattaccggcatcattctttctgaaatacagaacggttctttttctttccgggatttttatacccgcaggattaagcggatttatcctgcttttatcgcggccgtgtcgctggcttcggtgattgcctctcaaatcttcctttacgaagatttcaaccaaatgcggaaaaccgtggagctttctgcggttttcttgtccaatatttatctggggtttcagcaggggtatttcgatttgagtgccgacgagaaccccgtactgcatatctggtctttggcggtagaggaacagtattacctcctgtatcctcttttgctgatattttgctgcaaaaaaaccaaatcgctacgggtgctgcgtaacatcagcatcatcctatttctgattttgactgccacatcgtttttgccaagcaggttttataccgacatcctcaaccaacccaatacttattacctttcgacactgaggtttcccgagctgttggcaggttcgctgctggcggtttacgggcaaacgcaaaacggcagacggcaaacagcaagcggaaaacggcagttgctttcattactctgcttcggcgcattgcttgcctgcctgttcgtgattgacaaacacaatccgtttatcccgggaatgaccctgctccttccctgcctgctgacggcactgcttatccggagtatgcaatacgggacacttccgacccgcatcctgtcggcaagccccatcgtatttgtcggcaaaatctcttattccctatacctgtaccattggatttttattgccttcgcccattacattacaggcgacaaacagctcggactgcctgccgtatcggcggttgccgcattgacggccggattttccctgttgagctattatttgattgaacagccgcttagaaaacggaagatgaccttcaaaaaggcatttttctgcctctatctcgccccgtccctgatacttgtcggttacaacctgtacgcaagggggatattgaaacaggaacacctccgcccgttgcccggcgcgccccttgctgcggaaaatcattttccggaaaccgtcctgaccctcggcgactcgcacgccggacacctgcggggttttctggattatgtcggcagccgggaagggtggaaagccaaaatcctgtccctcgattcggagtgtttggtttgggtggatacgacactggcagacaacccgttatgtcgaaaataccgggatgaagttgaaaaagccgaagccgttttcattgcccaattctatgatttgaggatgggcggccagcccgtgccgagatttgaagcgcaatccttcctaatacccgggttccaagcccgattcagggaaaccgtcaaaaggatagccgccgtcaaacccgtctatgtttttgcaaacaacacatcaatcagccgttcgcccctgagggaggaaaaattgaaaagatttgccgcaaaccaatatctccgccccattcaggctatgggcgacatcggcaagagcaatcaggcggtctttgatttggttaaagatatccccaatgtgcattgggtggacgcacagaaatacctgcctaaaaacacggtcgaaatacacggacgctatctttacggcgaccaagaccacctgacctatttcggttcttattatatggggcgggaatttcacaaacacgaacgcctgcttaaatcttcccgcggcggcgcattgcagtag

SEQ ID NO: 44 - DNA, 1056 bp, N. meningitidis
atgaagctcaaaatagacattgcaaccaacaacttcaaacacggcggcggcacggaacgctacacattggatttggtaaagggtctgaacagacaaaacatcacaccggccgtttatgcgacgaaatttgatcacggcattcctgaatacgccatgatcgaaccccatcttgtcgatcaacaccggacgctgaaaaaactacgctcattcctcttttcaagccggctcactcaaaccagaaaaaacagtgccgccaaactgattgcctgccaccacgccgattacgccgacctcctcatctgcggcggcacacacttgggctacctgcaccatatggcgcaaaaaccgaacctgctcgaccgcctcgccatacgccgtaaccgcagcaactacgccaccgccaaactgattatggcgcattcccatatgatgcggcgcgaactggtcggactgtacggcgttccccctgaaagaatccaagtcgcccccccccccgcagatacggaacgcttcttcccgcaacccggagaaactgccgacctgcgcgccaaatacggctttgccgactatgaaaccgttttcctgttcccatcgaccggccacacgcgcaaaggtctggaactgcttgccgactttttcgaacataccggcctgcccgtcaagctcgccgttgtcggctccccgctcccccgccctatgaaaaacgtcgtcgggctgggcttctgcaccgatatgcccgaactctaccgcgccgccgactttaccattatggcctccctgtacgaacccttcgggctggtcggtgtcgaatccgtcctatgcggcacacgcgtcgtcctctccgaaaacatggcatgtacagaggtcatgaacgaagaagccggattctttttctcacgccaaaacccggaaaccctggcgcaagccgttgcccaagccgtcagccttaaaaaacagggcggacaccgcctgtccgacccgatgcgtgcactgaactacaacccgtccctgtcgcaccacatcgaccgactgaccgacatgctggcatccgtataa